SEQUENCE LISTING (24 sequences)

SEQ ID NO: 1 - DNA, 477 bp, Artificial Sequence
  Synthetic DELTA-11S nucleotide sequence
atgggccatcatcatcatcatcatcatcatatggtttttacacttgaagattttgtgggtgattgggaacaaactgctgcatataatttagatcaagttttagaacagggtggagtgagttctcttttacaaaatcttgcagtctcagttacaccaatacaaagaatagttagaagtggagaaaatgcattaaagatcgatatacatgtaataattccttatgagggactatcagcagaccaaatggcacaaattgaggaagtattcaaagttgtatatccagtagacgatcatcactttaaagtaatattaccttatggaactttagtaattgatggtgtaacaccaaatatgttaaattattttggtagaccatacgaagggattgcagtttttgatggaaagaaaataaccgtaacaggcactttgtggaatggaaataaaataatagatgaaaggttaattacacctgattaa

SEQ ID NO: 2 - protein, 158 aa, Artificial Sequence
  Synthetic DELTA-11S amino acid sequence
MGHHHHHHHHMVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPD

SEQ ID NO: 3 - DNA, 33 bp, Artificial Sequence
  Synthetic Beta-9 nucleotide sequence
ggctccatgctgttccgagtaaccatcaacagt

SEQ ID NO: 4 - protein, 11 aa, Artificial Sequence
  Synthetic Beta-9 amino acid sequence
GSMLFRVTINS

SEQ ID NO: 5 - DNA, 33 bp, Artificial Sequence
  Synthetic BETA-10 nucleotide sequence
gtgagcggctggcggctgttcaagaagattagc

SEQ ID NO: 6 - protein, 11 aa, Artificial Sequence
  Synthetic BETA-10 amino acid sequence
VSGWRLFKKIS

SEQ ID NO: 7 - DNA, 261 bp, Artificial Sequence
  Synthetic Beta-9-G nucleotide sequence
atgggctccatgctgttccgagtaaccatcaacagtggaggtggatccggtggtggagggagcacctacaaacttgtcattaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcaccgtaacggaaggtggcggtagccatcaccaccatcaccactga

SEQ ID NO: 8 - protein, 86 aa, Artificial Sequence
  Synthetic Beta-9-G amino acid sequence
MGSMLFRVTINSGGGSGGGGSTYKLVINGKTLKGETTTEAVDAATAEKVFKQYANDNGVDGEWTYDDATKTFTVTEGGGSHHHHHH

SEQ ID NO: 9 - DNA, 264 bp, Artificial Sequence
  Synthetic G-Beta-9 nucleotide sequence
atgggccatcatcatcatcatcatggtggagggagcacctacaaacttgtcattaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcaccgtaacggaaggtggcggtagcggtggcggaggctctggctccatgctgttccgagtaaccatcaacagttaa

SEQ ID NO: 10 - protein, 87 aa, Artificial Sequence
  Synthetic G-Beta-9 amino acid sequence
MGHHHHHHGGGSTYKLVINGKTLKGETTTEAVDAATAEKVFKQYANDNGVDGEWTYDDATKTFTVTEGGGSGGGGSGSMLFRVTINS

SEQ ID NO: 11 - DNA, 321 bp, Artificial Sequence
  Synthetic Beta-9-G-Beta-9 nucleotide sequence
atgggctccatgctgttccgagtaaccatcaacagtggaggtggatccggtggtggagggagcacctacaaacttgtcattaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcaccgtaacggaaggaggaggatctggaggtggatccggctccatgctgttccgagtaaccatcaacagtggcggaagccatcatcaccaccatcatcaccattga

SEQ ID NO: 12 - protein, 106 aa, Artificial Sequence
  Synthetic Beta-9-G-Beta-9 amino acid sequence
MGSMLFRVTINSGGGSGGGGSTYKLVINGKTLKGETTTEAVDAATAEKVFKQYANDNGVDGEWTYDDATKTFTVTEGGGSGGGSGSMLFRVTINSGGSHHHHHHHH

SEQ ID NO: 13 - DNA, 867 bp, Artificial Sequence
  Synthetic Beta-10-RBD nucleotide sequence
atgcggcttccgggtgcgatgccagctctggccctcaaaggcgagctgctgttgctgtctctcctgttacttctggaaccacagatctctcagggcggtggagtgagcggctggcggctgttcaagaagattagcggtggcggaggatctggaggtggatccagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcggcggaagccatcatcaccaccatcatcaccattga

SEQ ID NO: 14 - protein, 288 aa, Artificial Sequence
  Synthetic Beta-10-RBD amino acid sequence
MRLPGAMPALALKGELLLLSLLLLLEPQISQGGGVSGWRLFKKISGGGGSGGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGGSHHHHHHHH

SEQ ID NO: 15 - DNA, 807 bp, Artificial Sequence
  Synthetic RBD-Beta-10 nucleotide sequence
atgttcgtcttcctggtcctgctgcctctggtctcctcacagagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcggcggaggaggatctggaggtggatccgtgagcggctggcggctgttcaagaagattagcggcggaagccatcatcaccaccatcatcaccattga

SEQ ID NO: 16 - protein, 268 aa, Artificial Sequence
  Synthetic RBD-Beta-10 amino acid sequence
MFVFLVLLPLVSSQRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGGGGSGGGSVSGWRLFKKISGGSHHHHHHHH

SEQ ID NO: 17 - DNA, 927 bp, Artificial Sequence
  Synthetic Beta-10-RBD-Beta-10 nucleotide sequence
atgcggcttccgggtgcgatgccagctctggccctcaaaggcgagctgctgttgctgtctctcctgttacttctggaaccacagatctctcagggcggtggagtgagcggctggcggctgttcaagaagattagcggtggcggaggatctggaggtggatccagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcggcggaggaggatctggaggtggatccgtgagcggctggcggctgttcaagaagattagcggcggaagccatcatcaccaccatcatcaccattga

SEQ ID NO: 18 - protein, 308 aa, Artificial Sequence
  Synthetic Beta-10-RBD-Beta-10 amino acid sequence
MRLPGAMPALALKGELLLLSLLLLLEPQISQGGGVSGWRLFKKISGGGGSGGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGGGGSGGGSVSGWRLFKKISGGSHHHHHHHH

SEQ ID NO: 19 - DNA, 3888 bp, Artificial Sequence
  Synthetic Beta-10-S nucleotide sequence
atgcggcttccgggtgcgatgccagctctggccctcaaaggcgagctgctgttgctgtctctcctgttacttctggaaccacagatctctcagggcggtggagtgagcggctggcggctgttcaagaagattagcggtggcggaggatctggaggtggatcctgcgtcaatctgacaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcaggacgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcccggaagcgccagcagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggaccctcctgaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggcccggaggcagcggaggctacatccccgaggcccctcgcgatggacaggcttacgtgcgcaaggacggcgagtgggtgctgctgagcaccttcctgggcggaagccatcatcaccaccatcatcaccattga

SEQ ID NO: 20 - protein, 1295 aa, Artificial Sequence
  Synthetic Beta-10-S amino acid sequence
MRLPGAMPALALKGELLLLSLLLLLEPQISQGGGVSGWRLFKKISGGGGSGGGSCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGSGGYIPEAPRDGQAYVRKDGEWVLLSTFLGGSHHHHHHHH

SEQ ID NO: 21 - DNA, 3828 bp, Artificial Sequence
  Synthetic S-Beta-10 nucleotide sequence
atgttcgtcttcctggtcctgctgcctctggtctcctcacagtgcgtcaatctgacaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcaggacgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcccggaagcgccagcagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggaccctcctgaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggcccggaggcagcggaggctacatccccgaggcccctcgcgatggacaggcttacgtgcgcaaggacggcgagtgggtgctgctgagcaccttcctgggcggaggaggatctggaggtggatccgtgagcggctggcggctgttcaagaagattagcggcggaagccatcatcaccaccatcatcaccattga

SEQ ID NO: 22 - protein, 1275 aa, Artificial Sequence
  Synthetic S-Beta-10 amino acid sequence
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGSGGYIPEAPRDGQAYVRKDGEWVLLSTFLGGGGSGGGSVSGWRLFKKISGGSHHHHHHHH

SEQ ID NO: 23 - DNA, 3948 bp, Artificial Sequence
  Synthetic Beta-10-S-Beta-10 nucleotide sequence
atgcggcttccgggtgcgatgccagctctggccctcaaaggcgagctgctgttgctgtctctcctgttacttctggaaccacagatctctcagggcggtggagtgagcggctggcggctgttcaagaagattagtggtggcggaggatctggaggtggatcctgcgtcaatctgacaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcaggacgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcccggaagcgccagcagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggaccctcctgaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggcccggaggcagcggaggctacatccccgaggcccctcgcgatggacaggcttacgtgcgcaaggacggcgagtgggtgctgctgagcaccttcctgggcggaggaggatctggaggtggatccgtgagcggctggcggctgttcaagaagattagcggcggaagccatcatcaccaccatcatcaccattga

SEQ ID NO: 24 - protein, 1315 aa, Artificial Sequence
  Synthetic Beta-10-S-Beta-10 amino acid sequence
MRLPGAMPALALKGELLLLSLLLLLEPQISQGGGVSGWRLFKKISGGGGSGGGSCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGSGGYIPEAPRDGQAYVRKDGEWVLLSTFLGGGGSGGGSVSGWRLFKKISGGSHHHHHHHH

(19) United States
(12) Patent Application Publication
(10) Pub. No.: US 2024/0003881 A1
(43) Pub. Date: Jan. 4, 2024

(54) SYSTEM AND METHOD FOR RAPID AND SENSITIVE DETECTION OF ANTI-PATHOGEN ANTIBODIES

(71) Applicant: THE GOVERNING COUNCIL OF THE UNIVERSITY OF TORONTO, Toronto (CA)

(72) Inventors: Zhong YAO, Toronto (CA); Igor STAGLJAR, Toronto (CA)

(73) Assignee: THE GOVERNING COUNCIL OF THE UNIVERSITY OF TORONTO, Toronto, ON (CA)

(21) Appl. No.: 18/039,485

(22) PCT Filed: Dec. 3, 2021
(86) PCT No.: PCT/CA2021/051733

§ 371 Date: May 30, 2023

Related U.S. Application Data
(60) Provisional application No. 63/121,689, filed on Dec. 4, 2020.

(51) Int. Cl. G01N 33/569; G01N 33/543

(52) U.S. Cl. CPC G01N 33/56983; G01N 33/56944; G01N 33/54386; G01N 2333/315; G01N 2333/165; G01N 2469/20

(57) ABSTRACT

A serological detection system and a serological method of detecting a specific immunoglobulin (Ig) isotype against a target antigen in a sample, the serological detection system comprising: (a) a first probe having a first tag, the first probe having general binding affinity for all Ig having the same isotype as the specific Ig isotype in the sample, (b) a second probe having a second tag, the second probe being the target antigen of the specific Ig isotype in the sample, (c) a third tag, the third tag having binding affinity for the first tag and for the second tag so as to form a reporter complex in the presence of the specific IgG antibodies, and (d) a suitable substrate that generates an optically detectable signal in the presence of the reporter complex. In aspects, the serological detection system and the serological method are quantifiable.

### FIELD OF TECHNOLOGY

The present disclosure relates to system and method for rapid and sensitive detection of anti-pathogen antibodies.

### BACKGROUND INFORMATION

Testing is crucial for combating the coronavirus disease 2019 (COVID-19) pandemic¹. Serological tests are used to determine the level of antibodies against severe acute respiratory syndrome coronavirus 2 (SARS-CoV-2) in blood². Their results reflect the disease progress or its history, as well as the immunity of a patient³, which is valuable information for diagnosis and disease management. Importantly, with the advent of different upcoming vaccines, serological testing is becoming a necessary tool for evaluating acquired immunity at both individual and population levels. In addition, serological testing is essential for epidemic studies and related policymaking.

Numerous serological assays have been developed⁴. Among them, lateral flow immunoassays (LFIAs) are rapid and easy to perform, and therefore have found use as point-of-care tests⁵. However, their lack of quantifiability, coupled with their relatively low sensitivity and specificity, limits their usage as standard and reliable tests to evaluate antibody titers^{6,7}. Alternatively, enzyme-linked immunosorbent assays (ELISAs) are standard quantitative serological methods displaying good sensitivity and specificity^{8,9}. They too, however, have notable shortcomings including long processing time (3-5 hours), tedious procedures (multiple wash-aspirate cycles) and extra steps involved in pre-processing of binding plates. Several chemiluminescent immunoassay (CLIA) platforms targeting COVID-19 have also been developed by companies such as Abbott¹⁰, DiaSorin¹¹, Roche¹² and Siemens¹³. These are highly automated assays suitable for measurement of large content samples and are characterized by good quantifiability and sensitivity¹³. However, the measurements require highly specialized and expensive instruments, which limit their widespread application.

Protein complementation assays (PCAs) are a strategy to detect protein-protein interactions (PPIs)^{14,16}. In this approach, a ‘sensor’ protein is split into two fragments, which are then fused to two candidate interacting proteins of interest. The binding of the two proteins of interest arranges the sensor fragments in a favourable position that allows them to reconstitute into a functional protein which can produce a detectable signal representative of the PPI¹⁶. Different sensors such as fluorescent proteins, transcription factors, proteases and more have been successfully used in various designs. Among them, split luciferases have been shown to have the advantages of high signal/noise ratio and rapid reconstitution, making them widely used 17-19.

However, the conventional strategy of splitting luciferase into two fragments has limitations. For instance, the relatively large size of the fragments may interfere with target protein folding or function and/or the interaction with partner molecules. The residual intrinsic affinity between the two luciferase fragments may also lead to increased background signal. A recently developed tri-part strategy circumvents these limitations by splitting NanoLuc® (NLuc), the brightest luciferase identified so far, into three fragments: two short peptides (β9 and β10 each containing 11 amino acids) and one 16 kDa fragment (Δ11S)^{26,21}.

### SUMMARY OF DISCLOSURE

The present disclosure relates to a liquid-phase serological system and assay for immunoglobulin of a specific isotype against a target antigen based on split tripart Nanoluciferase (tNLuc) that can be performed directly with patient sera. The systems and assays display quantifiability and sensitivity comparable to ELISA, are rapid/easy to perform, cost-efficient and produce readouts highly consistent with neutralizing antibody tests, strongly supporting its potential value in COVID-19 diagnosis and disease and vaccination management.

In one embodiment, the present disclosure relates to a serological detection system for detecting immunoglobulins (Ig) of a specific isotype against a target antigen (specific Ig isotype) in a sample, the serological detection system comprising: (a) a first probe having a first tag, the first probe having general binding affinity for all Ig of the same isotype as the specific Ig isotype in the sample, (b) a second probe having a second tag, the second probe being the target antigen of the specific Ig isotype in the sample, (c) a third tag, the third tag having binding affinity for the first tag and for the second tag so as to form a reporter complex in the presence of the specific IgG antibodies, and (d) a suitable substrate that generates an optically detectable signal in the presence of the reporter complex.

In one embodiment of the serological detection system, the first tag is included at the N-terminus of the first probe, at the C-terminus of the first probe or at both the N-terminus and the C-terminus of the first probe.

In another embodiment of the serological detection system, the second tag is included at the N-terminus of the second probe, the C-terminus of the second probe or both the N-terminus and the C-terminus of the second probe.

In another embodiment of the serological detection system, the first probe recognizes immunoglobulin G (IgG) without cross-reactivity with other Ig isotypes.

In another embodiment of the serological detection system, the first probe is a protein G, or a suitable domain thereof.

In another embodiment of the serological detection system, the first probe is a protein G of a Streptococcus sp.

In another embodiment of the serological detection system, the first probe is a C1, a C2 or a C3 domain of protein G of a Streptococcus sp.

In another embodiment of the serological detection system, the Streptococcus sp. is Streptococcus sp. G148.

In another embodiment of the serological detection system, the first probe recognizes immunoglobulin M (IgM) without cross-reactivity with other Ig isotypes or the first probe recognizes immunoglobulin A (IgA) without cross-reactivity with other Ig isotopes.

In another embodiment of the serological detection system, the third tag is a Δ11S peptide of a nanoluciferase (NanoLuc).

In another embodiment of the serological detection system, the first probe is a protein G or a suitable protein G domain for general detection of IgGs in the sample, and the first tag is a β9 peptide of a nanoluciferase (NanoLuc), the second probe is the target antigen of the specific IgG isotype, the second tag is a β10 peptide of NanoLuc, the third tag is a Δ11S of NanoLuc or a fragment thereof, and the substrate is a reagent that generates the optically detectable signal in the presence of NanoLuc.

In another embodiment of the serological detection system, the first probe is a protein G or a suitable protein G domain for general detection of IgGs in the sample, and the first tag is a β10 peptide of a nanoluciferase (NanoLuc), the second probe is the target antigen of the specific IgG isotype, the second tag is a 139 peptide of NanoLuc, the third tag is a Δ11S of NanoLuc or a fragment thereof, and the substrate is a reagent that generates the optically detectable signal in the presence of NanoLuc.

In another embodiment of the serological detection system, the 139 peptide is included at the N-terminus, the C-terminus or both the N-terminus and the C-terminus of the first probe or of the second probe.

In another embodiment of the serological detection system, the β10 peptide is included at the N-terminus, the C-terminus or both the N-terminus and the C-terminus of the first probe or of the second probe.

In another embodiment of the serological detection system, the 139 peptide is included at the N-terminus of the protein G or of the suitable protein G domain, and the β10 peptide is included at both the N-terminus and the C-terminus of the protein characteristic of the target.

In another embodiment of the serological detection system, the first probe is an anti IgM antibody for general detection of IgMs in the sample, and the first tag is a β9 peptide of a nanoluciferase (NanoLuc), the second probe is the target antigen of the specific IgM isotype, the second tag is a β10 peptide of NanoLuc, the third tag is a Δ11S of NanoLuc or a fragment thereof, and the substrate is a reagent that generates the optically detectable signal in the presence of NanoLuc.

In another embodiment of the serological detection system, the target antigen is from an infectious pathogen.

In another embodiment of the serological detection system, the pathogen is a bacterium, a fungus, a virus, a yeast, algae or a protozoan.

In another embodiment of the serological detection system, the pathogen is severe acute respiratory syndrome coronavirus 2 (SARS-CoV-2).

In another embodiment of the serological detection system, the second probe is a spike (S) protein of SARS-CoV-2 or a fragment thereof.

In another embodiment of the serological detection system, the second probe is a receptor binding protein of a spike protein of SARS-CoV-2.

In another embodiment of the serological detection system, the target antigen is an autoimmune antigen and the specific Ig isotype are autoantibodies.

In another embodiment of the serological detection system, strength of the optically detectable signal correlates with the amount of the specific Ig isotype in the sample, thereby quantitating the specific Ig isotype against the target antigen in the sample.

In another embodiment, the present disclosure relates to a serological method of detecting the presence of specific Ig isotype against a target antigen in a sample of a subject, the method comprising: (a) reacting the sample with the serological system according to an embodiment of the present disclosure and (b) exposing the sample to an apparatus that detects the optically detectable signal in the sample, wherein detection of the optically detectable signal in the sample is indicative of the presence of the specific Ig isotype against the target antigen in the sample.

In one embodiment of the serological method, the optically detectable signal has a strength, and the strength of the optically detectable signal is quantifiable and correlates with the amount of the specific Ig isotype against the target antigen in the sample.

In another embodiment of the serological method, strength of the optically detectable signal detected in the sample is compared to strength of optically detectable signal detected from a control sample devoid of the specific Ig isotype (negative control), and when the strength of optically detectable signal from the sample is greater than the strength of the optically detectable signal from the negative control is indicative of the presence of the specific Ig isotype in the sample.

In another embodiment of the serological method, when the specific Ig isotype is detected in the sample, the method further comprises treating the subject fora disorder associated with the target antigen.

In another embodiment of the serological method, the target antigen is from an infectious pathogen and wherein pathogen is a bacterium, a fungus, a virus, a yeast, algae or a protozoan.

In another embodiment of the serological method, the pathogen is severe acute respiratory syndrome coronavirus 2 (SARS-CoV-2).

In another embodiment of the serological method, the second probe is a spike (S) protein of SARS-CoV-2 or a fragment thereof.

In another embodiment of the serological method, the second probe is a receptor binding protein of a spike protein of SARS-CoV-2.29.

In another embodiment of the serological method, the target antigen is an autoimmune antigen and the specific Ig isotype are autoantibodies.

In another embodiment of the serological method, the strength of the optically detectable signal is plotted against antibody concentration on a concentration curve.

In another embodiment, the present disclosure relates to a serological diagnostic test of a disease in a subject comprising: (a) reacting a sample taken from the subject with (i) a first probe having a first tag, the first probe having general binding affinity to one immunoglobulin (Ig) isotype in the sample without cross-reacting with other Ig isotypes, and (ii) a second probe having a second tag, the second probe being an antigen characteristic of said disease having binding affinity to specific Ig isotype in the sample, (b) reacting the sample with (i) a third tag having binding affinity for the first tag and for the second tag so as to form a reporter complex in the presence of the specific IgG antibodies, and (ii) a suitable substrate that generates an optically detectable signal in the presence of the reporter complex, and (c) exposing the sample to an apparatus that detects the optically detectable signal in the sample, wherein detection of the optically detectable signal in the sample is indicative of a positive test of the disease, and absence of optically detectable signal being indicative of a negative test of the disease.

In one embodiment of the serological diagnostic test, the optically detectable signal is quantifiable.

In another embodiment of the serological diagnostic test, strength of the optically detectable signal detected in the sample is compared to strength of optically detectable signal detected from a control sample devoid of the specific Ig isotype(negative control), and when the strength of optically detectable signal from the sample is greater than the strength of optically detectable signal from the negative control is indicative of a positive test and when the strength of optically detectable signal from the sample is equal or lower than the strength of optically detectable signal from the negative control is indicative of a negative test.

In another embodiment of the serological diagnostic test, the test is followed by treating the subject for the disease only when the test is positive.

In another embodiment of the serological diagnostic test, the first tag is included at the N-terminus, the C-terminus or both the N-terminus and the C-terminus of the first probe.

In another embodiment of the serological diagnostic test, the second tag is included at the N-terminus, the C-terminus or both the N-terminus and the C-terminus of the second probe.

In another embodiment of the serological diagnostic test, the first probe is a protein G or a suitable domain thereof.

In another embodiment of the serological diagnostic test, the first probe is a protein G of a Streptococcus sp.

In another embodiment of the serological diagnostic test, the first probe is a C1, C2 or a C3 domain of protein G of a Streptococcus sp.

In another embodiment of the serological diagnostic test, the Streptococcus sp. is Streptococcus sp. G148.

In another embodiment of the serological diagnostic test, the first probe specifically recognizes immunoglobulin M (IgM) without cross-reactivity with other Ig isotypes or recognizes immunoglobulin A (IgA) without cross-reactivity with other Ig isotopes.

In another embodiment of the serological diagnostic test, the third tag is a Δ11S peptide of a nanoluciferase (NanoLuc).

In another embodiment of the serological diagnostic test, the first probe is a protein G or a suitable domain of the protein G for general detection of IgG in the sample and the first tag is a β9 peptide of a nanoluciferase (NanoLuc), the second probe is a protein marker characteristic of the disease, the second tag is a β10 peptide of NanoLuc, the third tag is a Δ11S of NanoLuc, and the substrate is a reagent that generates a quantifiable optically detectable signal in the presence of NanoLuc.

In another embodiment of the serological diagnostic test, the first probe is a protein G or a suitable domain of the protein G for general detection of IgG in the sample and the first tag is a β9 peptide of a nanoluciferase (NanoLuc), the second probe is a protein marker characteristic of the disease, the second tag is a β10 peptide of NanoLuc, the third tag is a Δ11S of NanoLuc, and the substrate is a reagent that generates a quantifiable optically detectable signal in the presence of NanoLuc.

In another embodiment of the serological diagnostic test, the 139 peptide is included at the N-terminus, at the C-terminus or both the N-terminus and the C-terminus of the first probe or the second probe.

In another embodiment of the serological diagnostic test, the β10 peptide is included at the N-terminus, at the C-terminus or both at the N-terminus and the C-terminus of the first probe or the second probe.

In another embodiment of the serological diagnostic test, the 139 peptide is included at the N-terminus of the G protein, and the β10 peptide is included at both the N-terminus and the C-terminus of the protein marker characteristic of the disease.

In another embodiment of the serological diagnostic test, the disease is an infectious disease caused by a pathogen, and the second probe is an antigenic protein from said pathogen.

In another embodiment of the serological diagnostic test, the pathogen is a bacterium, a fungus, a virus, a yeast, algae or a protozoan.

In another embodiment of the serological diagnostic test, the infectious disease is COVID-19 and the pathogen is a severe acute respiratory syndrome coronavirus 2 (SARS-CoV-2).

In another embodiment of the serological diagnostic test, the second probe is a spike (S) protein of SARS-CoV-2.

In another embodiment of the serological diagnostic test, the second probe is a receptor binding protein of a spike protein of SARS-CoV-2.

In another embodiment of the serological diagnostic test, the disease is an autoimmune disease, and the second probe is an autoantigen having specific binding affinity to autoimmune IgG antibodies of said autoimmune disease.

In another embodiment of the serological diagnostic test, the sample is a bodily fluid sample of the subject.

In embodiments of the serological system, the serological method and the serological diagnostic test according to any one of the above embodiments, the subject is a human.

### BRIEF DESCRIPTION OF THE DRAWINGS

The following figures illustrate various aspects and preferred and alternative embodiments.

FIGS. 1A-1E. tNLuc assay for detecting α-SARS-CoV-2 antibody. 1A. Schematic workflow of the tNLuc assay. 1B. Scan of all probe formats/combinations in the tNuc assay using CR3022 Ab (2 μg/mL). Results are presented as a heatmap showing RLU values. 1C-1D. CR3022 at different concentrations was tested with the β9-G together with β10-S(C) or β10-Sβ10 (1D) probes. Human IgG, a mouse monoclonal Ab, and rabbit polyclonal Abs were used as controls. 1E. Comparison of β10-S and β10-Sβ10 affinities in the tNLuc system. The average Kd values of four experiments for each probe are presented as a bar graph. P value was calculated using a two tailed t-test.

FIGS. 2A-2E. Inhibitory effect of additional IgG on tNLuc assay. 2A. Dose response of IgG inhibition. Different amounts of human IgG as indicated were applied to samples containing 2 μg/mL CR3022 followed by analysis with the tNLuc assay. 2B. Inhibition kinetics of IgG in the assay was examined with different doses of CR3022, without IgG or in presence of human IgG at 25, 50 or 100 μg/mL, roughly the amounts in human serum at a 1:200 dilution. 2C. To mimic serum samples, different amounts of CR3022 (100, 33, 11 or 3.7 μg/mL) were spiked into buffer containing background human IgG at concentrations of 5, 10 or 20 mg/mL. Each sample was serially diluted and then analyzed with the tNLuc assay. Sums of luminescence readings at 1:300, 1:900 and 1:2700 for each sample are presented. 2D. Serially diluted CR3022 (100, 50, 25, 12.5, and 6.25 μg/mL) was spiked into serum samples (n=7, C1—C7 labeled with different colors and shapes) collected before the pandemic. In blank samples (gray circle), CR3022 was tested in buffer without mixing with serum. Each sample was tested and analyzed as in 2C. Basal value (baseline) was calculated as the mean of the seven serum samples without CR3022 spiking. The line of standard deviation (SD)×3 is highlighted as the limit of detection. Recovery is calculated as the percentage signal of a sample divided by the corresponding blank sample and is plotted in 2E. Data shown here are a representative result of two independent experiments with similar results.

FIGS. 3A-3B. Detection of α-SARS CoV-2 antibody in serum samples with the tNLuc assay. 3A. Samples included 7 collected before the pandemic and 82 from verified active or convalescent COVID-19 patients collected at different times after symptom onset. These were serially diluted as indicated and subjected to tNLuc testing. CR3022 (0.4 mg/mL in stock) was used as positive control. The level of anti-SARS CoV-2 antibodies at each dilution measured using tNLuc. Data shown in 3A are a representative result of two independent experiments with similar results. The dashed lines representing pre-pandemic samples are found next to the baseline (x axis) and they are obscured by the baseline. 3B. The overall antibody signal in each sample was calculated by summation of luminescence signals at dilutions 1:300, 1:900, and 1:2700. Samples are categorized in groups based on time elapsed between symptom onset and sample collection, and their distribution is presented in violin plots. The central dashed lines represent medians.

FIG. 4A-4D. Comparison of tNLuc assay with ELISA and neutralizing antibody assays. 4A. The same samples in FIG. 3 were tested using ELISA and the results are compared with tNLuc using scatter plot. 4B-4D. Eighty sera were subject to neutralizing antibody tests: sVNT (4B), PRNT50 (4C) and PRNT90 (4D). R and P-value were obtained from two-tailed Pearson correlation analysis. Source data for FIGS. 4A to 4D are presented in Table 1.

FIG. 5. Schematic of tNLuc probes.

FIGS. 6A-6C. Computational simulation of inhibitory effects of IgG on the tNLuc assay. 6A. Virtual samples containing CR3022 (3.7, 11, 33 and 100 μg/mL) in the presence of IgG at different levels (5, 10, and 20 mg/mL) were serially diluted as indicated. Their tNLuc signals at each dilution endpoint were calculated using the chemical kinetics model derived from the experimental data shown in FIG. 2B. 6B. Theoretical recovery rates of samples in 6A at different dilutions were calculated by dividing the calculated reading of a virtual sample in 6A by the corresponding virtual sample not containing IgG. 6C. The overall tNLuc signal of each virtual sample was calculated by luminescence summation of the calculated signals at the 1:300, 1:900 and 1:2700 dilutions obtained in 6A. Overall recovery rates are presented inset.

FIGS. 7A-7B. 7A. tNLuc-IgM assay for detecting anti-antigen IgM antibodies. 7B. Anti-S protein IgM levels in the sera of COVID-19 patients were evaluated using tNLuc-IgM assay presented herein. The results were plotted against days of symptom onset.

FIGS. 8A-8B. Detection of α-SARS CoV-2 antibody in serum samples with the tNLuc assay. 8A. 70 serum samples collected from COVID-19 patients or convalescents were analyzed in two independent replicates using the tNLuc assay. The two sets of data are presented as a scatter plot. R and P values were obtained from two tailed Pearson analysis. 8B. tNLuc luminescence signal of 80 samples plotted against the time of sample collection.

### DETAILED DISCLOSURE

### Definitions

In this specification and in the claims that follow, reference will be made to several terms that shall be defined to have the meanings below. All numerical designations, e.g., dimensions and weight, including ranges, are approximations that typically may be varied (+) or (−) by increments of 0.1, 1.0, or 10.0, as appropriate. All numerical designations may be understood as preceded by the term “about”.

The term “about,” particularly in reference to a given quantity, is meant to encompass deviations of plus or minus five percent.

The term “animal” includes humans and other animals.

The term sample includes body fluid. The term “body fluid”, as used herein, includes blood, serum, plasma, urine, cerebrospinal fluid, saliva and any other body fluid that includes IgGs.

Throughout this specification and the claims, the terms “comprise,” “comprises,” and “comprising” are used in a non-exclusive sense, except where the context requires otherwise. Likewise, the terms “include”, “has” and their grammatical variants are intended to be non-limiting, such that recitation of items in a list is not to the exclusion of other like items that can be substituted or added to the listed items. “Consisting essentially of” when used to define systems, compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers. “Consisting of” shall mean excluding more than trace elements of other ingredients and substantial method steps for using the systems of the present disclosure. Embodiments defined by each of these transition terms are within the scope of this disclosure.

The term “NT” as used herein refers to the N-terminal portion of a protein.

The term “CT” is used to refer to the C-terminal portion of a protein.

By “isolated” is meant, when referring to a polypeptide, that the indicated molecule is separate and discrete from the whole organism with which the molecule is found in nature or is present in the substantial absence of other biological macro molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

In this disclosure, the pathogen may be a human pathogen or an animal pathogen. The pathogen includes a bacterium, a fungus, a virus, a yeast, algae or a protozoan. In some embodiments, the pathogen is selected from the group consisting of Bacillus anthracis, Bordetella pertussis, Borrelia spp., Brucella spp., Chlamydia spp., Clostridium botulinum, Clostridium tetani, Corynebacterium diphtheriae, Enterobactereciae, Escherichia coli, Haemophilus influenza, Helicobacter pylori, Hemophilus spp., Klebsiella spp., Streptococcus pneumonia, Legionella pneumophila, Listeria monocytogenes, Mycobacterium tuberculosis, Mycoplasma spp., Neisseria gonorrhoeae, Neisseria meningitidis, Pseudomonas spp., Ricketsia spp., Salmonella spp., Shigella spp., Staphylococcus spp., Streptococci spp., Vibrio cholera, Yersinia spp., Adenovirus species, corona virus species, CCHF virus, Cytomegalovirus, Dengue virus, Ebola virus, Epstein-Barr virus, SARS-CoV-2, Hepatitis A virus, Hepatitis B virus, Hepatitis C virus, Herpes simplex viruses, HIV, HTLV, Human Herepes virus 6-8, Influenza virus, Measles virus, Mumps virus, Polio virus, Rabies virus, Rubella virus, SARS and associated coronaviruses, Respiratory Syncytial virus, Varicella Zoster virus, West Nile Virus, Yellow Fever virus, Zika virus, Plasmodium spp., and agents of endemic mycoses.

Overview

The present disclosure relates to a novel approach for detecting the presence of specific immunoglobulin isotypes against specific antigens in a sample. In alternative embodiments, the specific immunoglobulin (Ig) isotype immunoglobulin G (IgG) or immunoglobulin M (IgM) or IgA. In aspects the specific Ig antibody is a-severe acute respiratory syndrome coronavirus 2 (SARS-CoV-2) IgG or IgM. In this approach, a tri-part split of a reporter protein is used as a sensor for detecting the presence of the specific anti-antigen antibody, such as α-SARS CoV-2 antibody.

Presented herein is a new serological system and assay for the detection of specific Ig antibodies against specific antigens with a quantifiability like that of ELISA but featuring great ease of use, more rapid detection and lesser requirement for specialized equipment. Furthermore, its readout shows tight correlation to neutralizing capability. Collectively these features suggest the system and methods of this disclosure have great value for use in clinical laboratories involved in pathogen testing. Additionally, the strategy employed in the assay described in this disclosure has the potential to be adapted for use in alternative diagnostic approaches to help combat infectious disorders or autoimmune diseases.

The antigens, in embodiments, are from infectious pathogens or from autoimmune disorders.

In one embodiment, the present disclosure is a serological detection system of a specific immunoglobulin isotype against a target antigen in a sample. In embodiment, the system includes: (a) a first probe having a first tag, the first probe having general binding affinity for the Ig isotype of the specific isotype in the sample, (b) a second probe having a second tag, the second probe being the antigen having specific binding affinity to the specific Ig isotype in the sample, (c) a third tag, the third tag having binding affinity for the first tag and for the second tag so as to form a reporter complex in the presence of the specific Ig isotype, and (d) a suitable substrate that generates an optically detectable signal in the presence of the reporter complex. In one embodiment, the specific Ig isotype is IgG. In another embodiment, the specific Ig isotype is IgM.

In embodiments, the present disclosure describes serological method of detecting the presence of a specific Ig isotype against a target of interest, which includes pathogen or antigens of an autoimmune disease in a sample of a subject, the method comprising: (a) reacting the sample with (i) a first probe having a first tag, the first probe having general binding affinity for the Ig isotype of the specific Ig isotype in the sample, and (ii) a second probe having a second tag, the second probe being an antigen having specific binding affinity to the specific Ig isotype in the sample, (b) reacting the sample with (i) a third tag having binding affinity for the first tag and for the second tag so as to form a reporter complex in the presence of the specific Ig isotype, and (ii) a suitable substrate that generates an optically detectable signal in the presence of the reporter complex, and (c) exposing the sample to an apparatus that detects the optically detectable signal in the sample, wherein detection of the optically detectable signal in the sample is indicative of the presence of-specific Ig isotype in the sample. In one embodiment, the specific Ig isotype is IgG. In another embodiment, the specific Ig isotype is IgM.

In some embodiments, the present disclosure relates to a serological diagnostic test of an infectious disease caused by a pathogen, or of an autoimmune disease in a subject comprising: (a) reacting a sample taken from the subject with (i) a first probe having a first tag, and (ii) a second probe having a second tag, the second probe being an antigen from the pathogen or from the autoimmune disease having specific binding affinity to a specific Ig isotype in the sample, the first probe having general binding affinity for the Ig isotype of the specific Ig isotype in the sample (b) reacting the sample with (i) a third tag having binding affinity for the first tag and for the second tag so as to form a reporter complex in the presence of the specific Ig isotype, and (ii) a suitable substrate that generates an optically detectable signal in the presence of the reporter complex, and (c) exposing the sample to an apparatus that detects the optically detectable signal in the sample, wherein detection of the optically detectable signal in the sample is indicative of a positive test, and absence of optically detectable signal being indicative of a negative test. In one embodiment, the specific Ig isotype is IgG. In another embodiment, the specific Ig isotype is IgM.

The first prole has binding affinity to any Ig of the same isotype of the specific lg. For example, if the specific Ig is IgG, then the first probe has general binding affinity to all IgGs, including the specific IgG. If the specific Ig is IgM, then the first probe has general binding affinity to all IgMs, including the specific IgM.

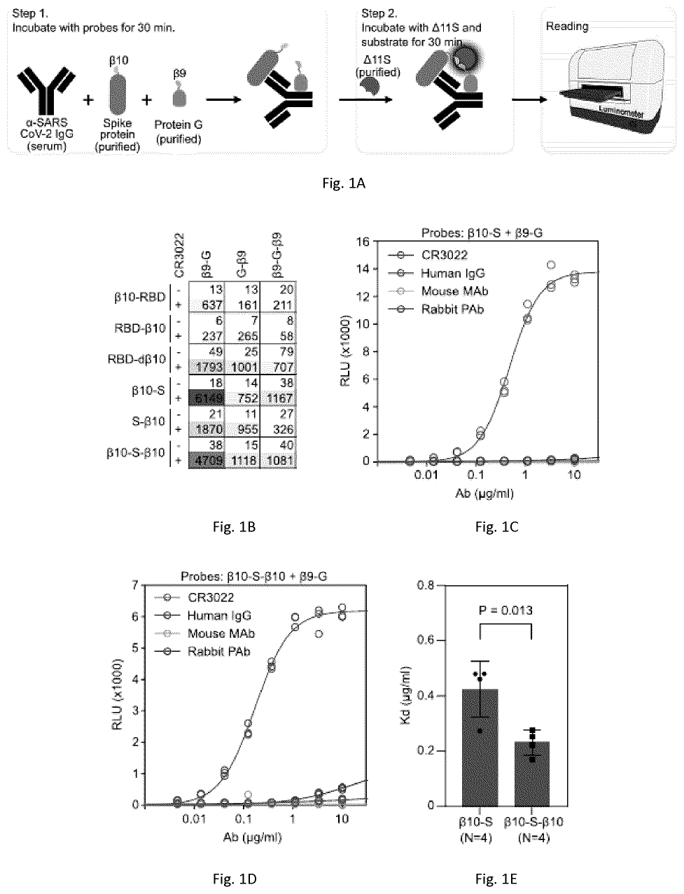

In embodiments, the serological systems and assays of the present disclosure are based on a splitting of NanoLuc (Nluc), the brightest luciferase identified so far, into three fragments or tags: two short peptides (β9 and β10 each containing 11 amino acids) and one 16 kDa fragment (Δ11S). The β9 and β10 tags are separately fused to a pair of probes. The first of these probes is suitable for general detection of one Ig isotype and has no cross-reactivity with other Ig isotypes. In one embodiment the Ig isotype is IgG and examples of the first probe include protein G of different streptococcal strains, or a domain of protein G, that exclusively binds to all isotypes of human IgG but does not bind to or cross-react with IgM, IgA or IgE. In another embodiment, the Ig isotype is IgM, and the first probe include is an IgM binder that specifically recognizes IgM and has no cross-reactivity with IgG, IgA or IgE molecules. Examples of IgM binders include antibodies that generally bind to IgM, such as those developed by NanoTag Biotechnologies (clone 2F2). The first probe is fused to a first tag, including the β9 peptide or the β10 peptide. The second probe is an antigen of the specific IgG isotype or IgM isotype and the antigen is of the disease being assayed. In embodiments, the disease is a COVID-19 infection, and the specific Ig is α-SARS CoV-2 antibody, and the second probe is made, for example, from the viral spike (S) protein (including the receptor binding domain, RBD) or nucleocapsid (N) protein. The S protein is responsible for virus binding to receptor and the target of neutralizing antibodies in patients. As illustrated in FIG. 1A, (i) diluted serum or plasma samples are mixed with the two probes and incubated for an effective amount of time; (ii) an aliquot of this mixture is combined with the third component Δ11S (i.e., the third tag) and substrate and incubated for another effective amount of time, followed by luminescence recording with an apparatus such as a luminometer. An optical signal will be generated when the antibody against the target of interest is present in the sample.

A commonly used protein G is from Streptococcus G148, and it contains multiple domains. Domains C1, C2 and C3 are responsible for IgG binding with slight difference in their specificity. Protein G from other Streptococcus stains (such as G43 and C40) contains two IgG binding domains which are correspondent to the C1 and C3 domain of the G148 strain. All of these domains are suitable for the systems and methods of this disclosure. In the examples below, C2 of the G148 domain is used. The C3 domain of G148 was also tested and it seems to behave similarly as C2.

In embodiments, the serological systems, tests and methods of the present disclosure may be used as a high-throughput screening technology for detecting the presence of IgG antibodies against a pathogen or against any other target of interest.

The systems and methods of the present disclosure can also be used to follow the efficacy of a treatment of infectious disorders or other diseases. For example, in the case of COVID-19, a patient can be tested for quantifiably amount of α-SARS CoV-2 antibody at different stages of the treatment using the systems, tests and methods of this disclosure. A reduction in the amount of α-SARS CoV-2 antibody being indicative of the efficacy of the COVID-19 treatment.

In order to aid in the understanding and preparation of the present disclosure, the following illustrative, non-limiting examples are provided.

### EXAMPLES

### Example 1

Plasmids and Recombinant Proteins.

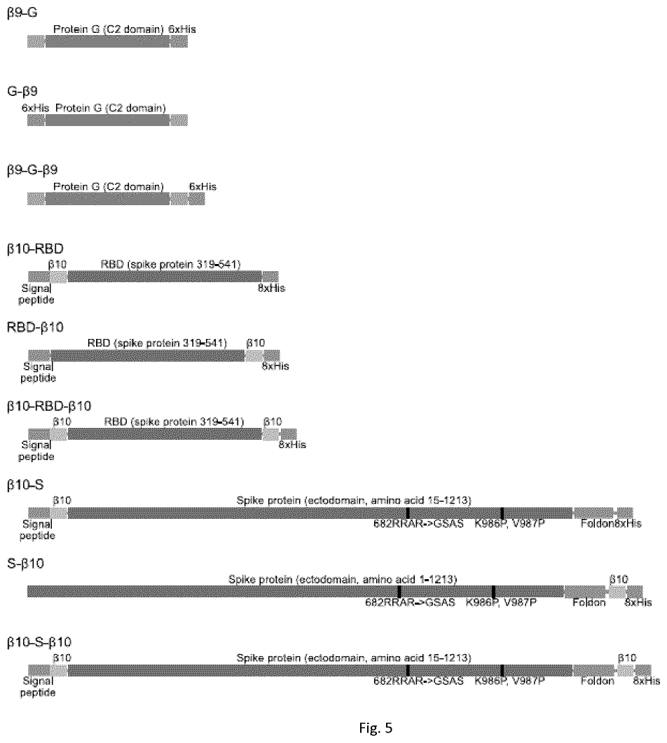

To create the general probe, the C2 domain of protein G of Streptococcus sp. (strain G148) was appended with the 89 tag and a 6xHis tag either at its N- or C-terminus (or both) (FIG. 5A). The cDNAs were cloned into pET16b by Gibson assembly. The plasmids were transformed into BL21-Gold (DE3) cells. For expression, the bacteria were grown at 37° C. until OD550 0.4-0.6, followed by further incubation at 22° C. for 4 hours in the presence of 0.2 mM IPTG. Bacterial cells were collected and lysed by sonication for further purification. Antibody specific probes were created by appending 810 tag to N— or C—, or both termini of SARS-CoV-2 spike protein (ectodomain) or its RBD 32. PDGFRB signal peptide was used as N-terminal leading sequence for N-tagged constructs to allow their correct secretion to extracellular space. PolyHis tag was added at their C-termini for purpose of purification. Several modifications were made to the S protein ectodomain probe similar to that previously reported: the foldon sequence was fused to the C-termini to promote trimerization; the furin cleavage site 682RRAR was mutated to GSAS; K986 and R987 were mutated to prolines to stabilize the prefusion conformation (FIG. 5A). The plasmids of antibody probes were transfected to HEK 293 cells by PEI and media of cultured cells were collected. Both general probes and antibody probe were purified with Ni Sepharose (Cytiva 17526801) according to the product manual. CR3022 was produced as described previously³³. Δ11S was constructed and purified as described²⁰. tNLuc assay. Patient sera or CR3022 were diluted in phosphate buffer saline supplemented with 0.1% Tween-20 and 0.1% fatty acid-free bovine serum albumin. In some cases, human IgG (Sigma 14506) was added to mimic human serum sample. The samples were mixed with probes at final concentration of 150 nM for general probes and 4 nM for antibody probes and were incubated at room temperature for 30 minutes. An aliquot of the mixture (5 μl of sera sample or 2 μl of CR3022 sample) was mixed with 9 volume of Nano-Glo buffer and substrate (Promega N1110) supplemented with recombinant Δ11S (final concentration 10 μg/ml) in 96 or 384 well reading plate. After another 30 minutes of incubation at room temperature, luminescence signals were recorded by a microplate luminometer. CR3022 and probe binding kinetics was mathematically fitted with GraphPad Prism 8 in an allosteric sigmoidal model. IgG inhibitory effects on CR3022 binding kinetics was fitted in an allosteric noncompetitive model:

$$\\ Y = \frac{{Y}_{max} {X}^{{n}_{x}}}{{X}^{{n}_{x}} ( 1 + \frac{{I}^{{n}_{i}}}{α {K}_{i}^{{n}_{i}}} ) + {K}_{m}^{{n}_{x}} ( 1 + \frac{{I}^{{n}_{i}}}{{K}_{i}^{{n}_{i}}} )}$$

in which Y is the luminescence signal, X is the CR3022 concentration, I is the IgG concentration, nₓ is the Hill coefficient for CR3022 and probe interaction, and ni is the Hill coefficient for probe and IgG interaction.

ELISA. ELISA was undertaken according to previously described^{8,9} with some modifications. SARS-CoV-2 spike antigen was immobilized to a 384 well LUMITRAC high binding plate (Greinor Bio-One 781074) by incubating 20 μl/well of S-810 protein (20 nM in PBS) at 37° C. for 1 hour. After blocking with blocking buffer (PBS supplemented with 0.1% Tween 20 and 3% skimmed milk) for one hour at room temperature, the plate was incubated with sera serially diluted in blocking buffer at room temperature for 2 hours. After three times of wash with PBST (PBS supplemented with 0.1% Tween 20), the plate was further incubated with a-human IgG antibody conjugated to horse radish peroxidase (Jackson ImmunoResearch 109-035-098, 1:50,000 in blocking buffer, 30 μl/well) at room temperature for 1 hour. After three times of final wash with PBST, Pico chemiluminescence substrate mixture (Thermo Scientific 3769, 30 μl/m1) was added to the plate and signals were recorded in a microplate luminometer. Data of serially diluted samples were analyzed with GraphPad Prism 8 by model fitting. The total signal was calculated as areas under curve (AUC) of the fitted curves.

sVNT. sVNT kit was purchased from Genscript and the assay was performed according to the manufacturer's instructions²⁹. Briefly, diluted serum samples were mixed with HRP-RBD at a 1:1 ratio and incubated at 37° C. for 30 minutes. An aliquot (100 μl) of the mixture was moved to a well of a test plate which was precoated with ACE2 provided by the manufacturer and was incubated at 37° C. for 15 minutes. After 4 times wash, signal was developed by incubation with TMB solution.

PRNT. PRNT was performed as described previously³³. Briefly, diluted serum samples were incubated with SARS-CoV-2 virus (50 PFU) in a CO₂ incubator for one hour. The mixture was then moved to a well of a 12-well plate cultured with Vero E6 cells (100% confluency) and incubated for one hour in a CO₂ incubator with rocking every 15 minutes. To each well 1.5 ml prewarmed (37° C.) overlay medium (MEM without phenol red but supplemented with 4% FBS, L-glutamine, nonessential amino acids, sodium bicarbonate and 1.5% carboxymethycellose) was added followed by incubation for 72 hours. The cells were then fixed with 10% formalin (neutral-buffered) and subsequently stained with crystal violet (0.5% solved in 20% ethanol). Plaques were counted and compared to negative control. A titre is recorded as the highest serum dilution resulting in 50% and 90% reduction in plaques compared with controls. Human serum samples Negative control sera were taken before the pandemic. All patients were diagnosed by SARS-CoV-2 RT-PCR from nasopharyngeal swabs. All samples are de-identified and enrolled through REB approved protocols, REB20-044c or REB 149-1994.

Statistics. Statistical analysis was performed using GraphPad Prism 8. The performance difference between β10-S and β10-s-β10 was analyzed an unpaired two-tailed t test. Correlation between tNLuc assay and other assays (ELISA, VNT or PRNT) was analyzed using Pearson product-moment correlation.

### Results

In the present design, the β9 and β10 tags are separately fused to a pair of probes which can respectively recognize an IgG molecule against SARS-CoV-2 at different sites. The first probe is generated by fusing the β9 tag to the C2 domain of protein G, which exclusively binds to all the isotypes of human IgG but not to IgM, IgA or IgE immunoglobulins²². The second probe is specific to antibodies that bind the SARS-CoV-2 spike (S) protein, the viral membrane protein responsible for host cell receptor binding²³ and which is also the target of most of the neutralizing antibodies found in patients²⁴. We generated two forms of this probe by fusing the β10 tag to either the ectodomain of the S protein or to its receptor binding domain (RBD). The assay itself is remarkably straightforward to perform and involves only two simple steps (FIG. 1A): (i) diluted serum or plasma samples are mixed with the two probes and incubated for minutes; (ii) an aliquot of this mixture is combined with the third component, Ails, and the luciferase substrate and incubated for another 30 minutes, followed by luminescence recording with a luminometer. The whole process occurs directly in the liquid phase and does not require any washing steps.

As the peptide tags (β9 and β10) can be fused either at the N- or C-terminus of the probes or at both positions (FIG. 5), we first sought to determine which combination of the various probes would result in the most signal with the least background. We tested all combinations of the probes with CR3022, an antibody that binds to the RBD of the SARS-CoV-2 S protein²⁵, and observed that all combinations produced signal, albeit to varying extents (FIG. 1B). This suggests that despite the fact that the probes bind to different domains of IgG, the molecules are sufficiently flexible to allow proximal localization of the two tags, enabling reconstitution of tNLuc into an active luciferase. Of all the combinations, two produced the highest signal-to-background: β9-G with 810-S and 89-G with 810-S-810. Further testing of these two combinations using different concentrations of CR3022 demonstrated typical dose response curves (FIG. D). However, based on the calculated Kd values using 810-S(0.42 μg/mL) and 810-S-810 (0.23 μg/mL), we predicted that β10-S-β10 would have greater potential to detect antibody at lower concentrations (FIG. 1E). Thus, the β9-G/β10-S-β10 probe pair was chosen to be used in the final assay.

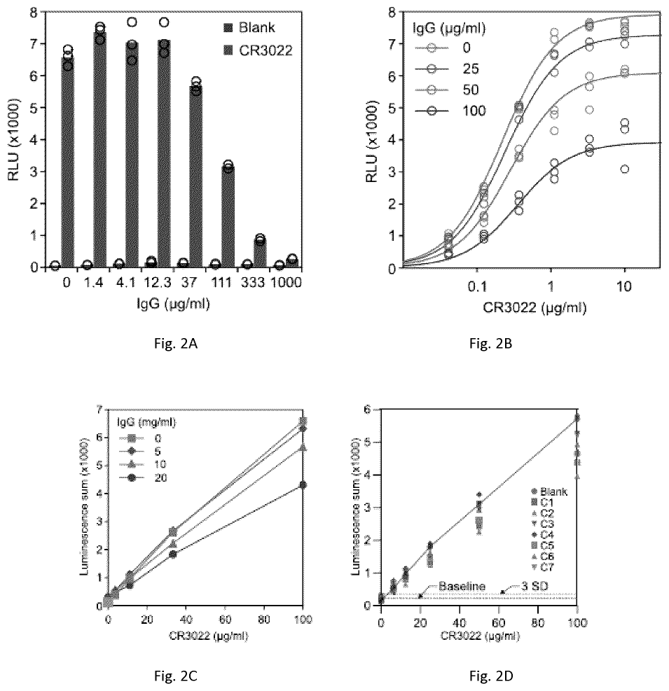

Human plasma contains a high concentration of IgG (4-22 mg/mL, median 11 mg/mL in serum)^{26,27}, the vast majority of which will not be specific for the SARS-CoV-2 S-protein. Since they can, however, bind the protein G probe, these “non-specific” antibodies will reduce the sensitivity of our assay. We investigated the effect of IgG interference on the assay by adding different amounts of human IgG into the reaction mixture and as predicted inhibition was indeed observed (FIG. 2A). The detailed kinetics of inhibition were further analyzed based on the dose response testing of CR3022 in the presence of different amounts of IgG (FIG. 2B). We performed mathematical analysis by fitting different inhibition models to these experimental data. A model of allosteric noncompetitive inhibition provided the best fit, as judged by visual comparison of the curve positions/shapes with the plotted data points (FIG. 2B) and the calculated R² (0.9911). The allosteric effect might be derived from the binding of protein G to two sites on an IgG molecule while the detailed molecular mechanism of noncompetitive inhibition merits further exploration.

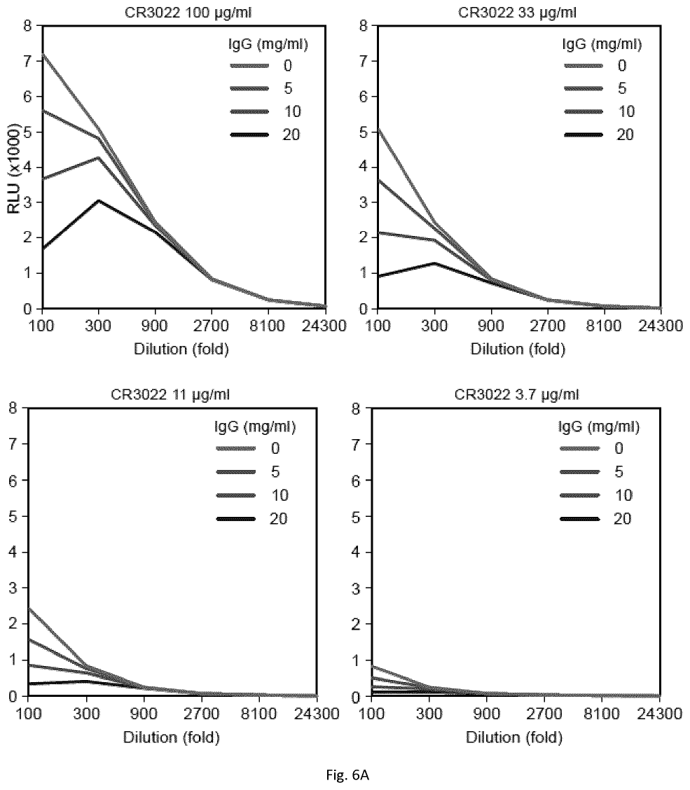

Binding parameters derived from the model (Ki=58.5 μg/mL, and Kd=0.22 μg/mL which is consistent with the results in FIG. 1E) suggest a considerable difference of the probe binding affinities toward CR3022 antibody and general IgG. We therefore reasoned that the inhibition can be eliminated or alleviated simply by dilution, a step also required to reduce nonspecific signal when performing ELISA on blood samples. This was supported by computational simulation of assays, using the obtained mathematical model, on virtual samples containing different concentrations of IgG and varying amounts of CR3022 (FIG. 6A). The recovery rates (the luminescence ratio of a sample and the correspondent control without additional IgG) at different dilution endpoints were calculated (FIG. 6B). At 1:100 dilution, the recovery rates for the samples of 20 mg/mL IgG (close to the upper limit in human serum) are around 20%. This was significantly improved by further dilution, reaching 47-60% at 1:300 dilution and 86-88% at 1:900 dilution. Reduced IgG brought even better recovery; samples containing 10 mg/mL IgG (close to the median IgG in human serum) showed ˜80% recovery at 1:300 dilution and 5 mg/mL of IgG (close to the lower limit of IgG in human serum) showed more than 90% recovery at 1:300 dilution.

Building on these results, we then adopted a strategy involving sample dilution to obtain more accurate measurements of the S protein specific antibodies. The overall tNLuc signal of a sample was calculated using the signal summation algorithm (luminescence sum of RLU values at 1:300, 1:900 and 1:2700 dilutions) to avoid parameter estimation, as required by other algorithms, while still maintaining a power similar to curve fitting²⁸. The signal at the lowest dilution endpoint (1:100) was excluded from the analysis to avoid the substantial signal interference caused by IgG under these conditions. Computational simulation (FIG. 6C) demonstrated good recovery; samples with medium and low IgG levels showed more than 80% recovery while samples with high IgG levels showed about 60% (for low CR3022 dose) to 70% (for high CR3022 dose) recovery. Performance was further evaluated experimentally with test samples containing varied doses of CR3022 and different concentrations of IgG (FIG. 2C); the results obtained were in good agreement with the computational prediction.

The performance was further evaluated via a spiking test with blank sera using seven serum samples collected before the COVID-19 pandemic. CR3022 was serially diluted and spiked into the matrix sera with concentrations of 100, 50, 25, 12.5, and 6.25 μg/mL. As before, tNLuc assays were then performed on these samples by measuring the luminescence produced at further dilutions of 300, 900, and 2700 times, and the overall signal of each sample was calculated as a luminescence sum (FIG. 2D). The signals show an apparent linear relationship with the antibody concentration, indicating the detection range can reach to at least 100 μg/mL. Calculation using the criterion of 3 times the standard deviation of the blank samples suggests the limit of detection of the assay is below 5 μg/mL. Most of the recovery rates are within the range of 80-120% (FIG. 2E), demonstrating the robustness of the assay. Since the samples contained serially diluted CR3022, the results also indicated a good dilution linearity. In addition, all inter-sample coefficients of variation of the groups with different amounts of spiked CR3022 were smaller than 20% demonstrating the good consistency of the assay.

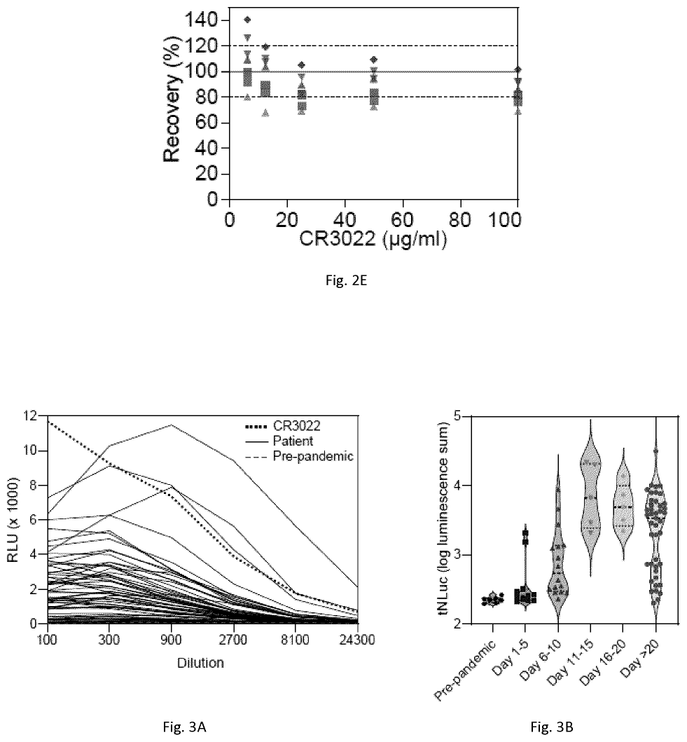

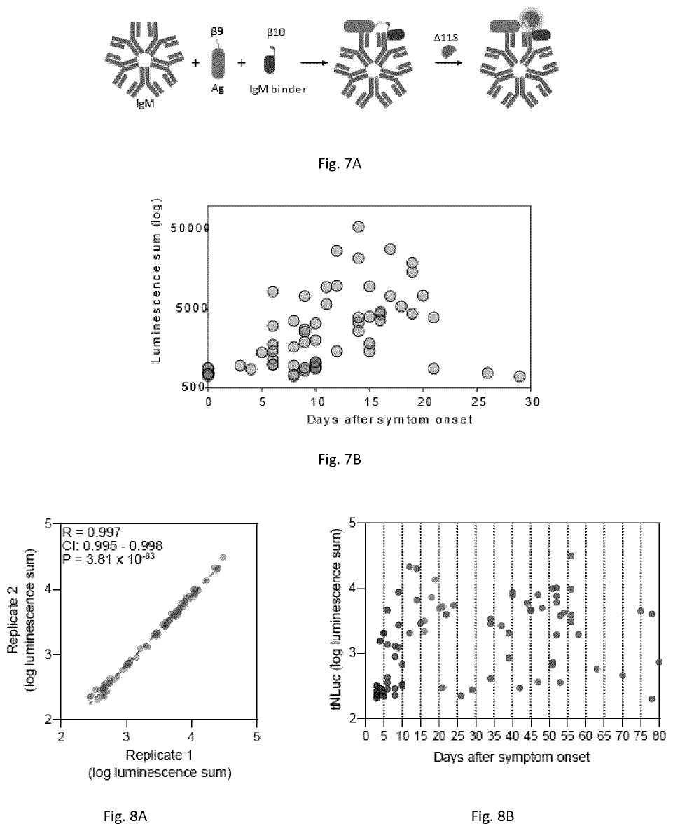

We then evaluated the tNLuc assay of the present disclosure with human serum samples. In addition to the above seven pre-pandemic sera, we also collected 82 serum samples from verified COVID-19 patients or convalescents across Canada, taken at different times (up to 80 days) after symptom onset. Again, the samples were serially diluted and analyzed using the tNLuc assay of the present disclosure. CR3022 was tested in parallel as a positive control. The samples displayed various signal amplitudes while those for pre-pandemic samples were close to baseline (FIG. 3A). As was done in FIG. 2C and FIG. 2D, the overall signal of the samples was calculated as luminescence summation of dilution points 1:300, 1:900, and 1:2700. A subset of the samples (n=70) was re-tested with tNLuc and comparison of the results obtained from these two independent tests showed a tight linear correlation (FIG. 8A), further demonstrating the consistency and reproducibility of the assay. Plotting the results against time of sample collection (FIG. 3b and FIG. 8B) also provided a rough overview of the kinetics of IgG production/levels in the context of COVID-19 progression in patients; only background signals were detected in pre-pandemic samples, antibodies were detected as early as 4-5 days after symptom onset, all the samples collected during days 11-20 produced high level signals and the majority of samples collected after day 20 still contained high level of antibodies although a very small fraction dropped to basal levels. The results showed excellent separation between controls and patient samples and the kinetics were consistent with multiple previous reports [3, 33-37], strongly suggesting good sensitivity and specificity of the tNLuc assay.

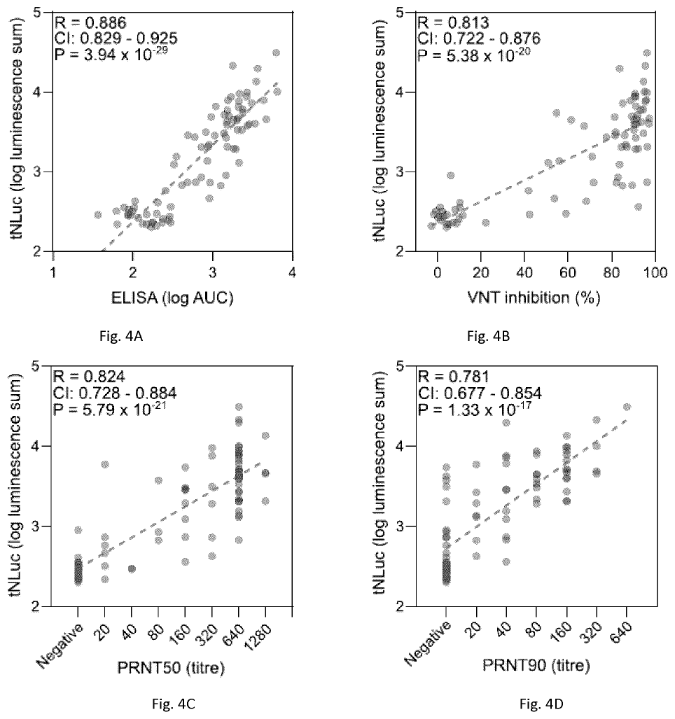

Finally, we compared the tNLuc assay with several other tests. Similar to the tNLuc assay, ELISA directly measures the antibodies specific to SARS-CoV-2 in blood samples. We performed the ELISA on all 14 samples using a common protocol⁹. The results demonstrated a high degree of correlation with those of the tNLuc assay (R²=0.783) (FIG. 4A), suggesting that the quantifiability and sensitivity of the tNLuc assay are similar to ELISA. In contrast to direct ELISA, neutralizing antibody assays measure only the antibodies responsible for antagonizing virus-receptor interaction. We therefore carried out two neutralization assays on 10 of the samples: the surrogate virus neutralization test (sVNT)²⁹ (FIG. 4B) and the plaque-reduction neutralization test (PRNT50 and 90)^{39,31}, the gold standard neutralization test, (FIGS. 4C, 4D). The results from each of these tests show high positive correlation with the tNLuc assay. Among them, the PRNT90 test produced results highly consistent with those of tNLuc (R 2=0.868). These observations suggest that the tNLuc assay provides a reliable indicator of the neutralization potential of a sample.

Presented herein is a new serological system and assay for the detection of antibodies against SARS CoV-2 with a quantifiability similar to that of ELISA but featuring with great ease of use, more rapid detection and lesser requirement for specialized equipment. Furthermore, the readout of the assay of the present disclosure shows tight correlation to neutralizing capability. Collectively these features suggest the system and methods of this disclosure have great value for use in clinical laboratories involved in COVID-19 testing. Additionally, the strategy employed in the assay described in this disclosure has the potential to be adapted for use in alternative diagnostic approaches to help combat other diseases, including autoimmune diseases, or other infectious diseases such as SARS, MERS and/or influenza.

TABLE 1
Comparison of tNLuc assay of the present disclosure to ELISA, sVNT and PRNT

| | tNLuc | | | | |
|---|---|---|---|---|---|
| | (Luminescence | ELISA | VNT | | |
| Dilution | sum) | (AUC) | (%) | PRNT50 | PRNT90 |
| OM8035 | 223 | 132.3 | | | |
| OM8066 | 232 | 177.2 | | | |
| OM8083 | 6490 | 2105 | | | |
| OM8087 | 3342 | 2305 | | | |
| CVD00768 | 1550 | 349.2 | 71.00342376 | 1:640 | 1:40 |
| CVD01207 | 292 | 148.8 | 42.17669654 | 1:40 | Negative |
| CVD01221 | 230 | 296.2 | 22.09987196 | Negative | Negative |
| CVD01286 | 264 | 116.8 | 0.339673913 | Negative | Negative |
| CVD01385 | 220 | 63.59 | 5.095108696 | 1:20 | Negative |
| CVD01569 | 246 | 281.1 | 1.637824475 | Negative | Negative |
| CVD01649 | 320 | 62.06 | −0.803461063 | 1:20 | Negative |
| CVD01715 | 210 | 195.7 | −2.822273074 | Negative | Negative |
| CVD01735 | 219 | 150 | 4.271548436 | Negative | Negative |
| CVD02040 | 2043 | 728.3 | 84.41403926 | ≥1:640 | 1:160 |
| CVD01182 | 4996 | 1602 | 89.21508035 | ≥1:640 | 1:160 |
| CVD01184 | 4669 | 2049 | 97.1574904 | ≥1:1280 | 1:160 |
| CVD01463 | 2066 | 1517 | 96.33802817 | ≥1:1280 | 1:160 |
| CVD01639 | 7838 | 1543 | 88.56613103 | ≥1:640 | 1:160 |
| CVD01641 | 3941 | 2854 | 91.34443783 | ≥1:640 | 1:160 |
| CVD01643 | 740 | 614.4 | 96.22248662 | 1:160 | 1:40 |
| CVD01645 | 364 | 297.6 | 92.28940217 | 1:160 | 1:40 |
| CVD01657 | 730 | 927.1 | 82.04573548 | 1:320 | Negative |
| CVD02063 | 5475 | 2388 | 54.63535229 | 1:160 | Negative |
| CVD02069 | 3775 | 2704 | 67.3053152 | 1:80 | Negative |
| CVD00130 | 901 | 1470 | 6.128232377 | Negative | Negative |
| CVD00240 | 256 | 238.3 | 9.883103082 | Negative | Negative |
| CVD00324 | 338 | 88.69 | 8.111937655 | Negative | Negative |
| CVD00336 | 289 | 36.64 | 4.279891304 | Negative | Negative |
| CVD00410 | 228 | 231.9 | 7.01381509 | Negative | Negative |
| CVD00829 | 285 | 93.72 | 11.61443245 | Negative | Negative |
| CVD00831 | 354 | 104.6 | 10.27126679 | Negative | Negative |
| CVD00868 | 411 | 205.8 | 10.3189007 | Negative | Negative |
| CVD01018 | 4568 | 4702 | 91.47247119 | ≥1:1280 | 1:320 |
| CVD01022 | 13582 | 3514 | 94.67349552 | ≥1:1280 | 1:160 |
| CVD01234 | 317 | 98.66 | 2.151088348 | Negative | Negative |
| CVD01314 | 1308 | 2138 | 50.78125 | ≥1:640 | 1:20 |
| CVD01339 | 21433 | 1779 | 95.48233696 | ≥1:640 | 1:320 |
| CVD01342 | 294 | 86.91 | −1.630434783 | Negative | Negative |
| CVD01384 | 1232 | 328.9 | 83.66168478 | 1:160 | 1:40 |
| CVD01433 | 8674 | 1693 | 90.79483696 | ≥1:640 | 1:80 |
| CVD01487 | 276 | 289.4 | 1.266996292 | Negative | Negative |
| CVD01581 | 4224 | 1506 | 93.72682324 | ≥1:640 | Negative |
| CVD01583 | 358 | 77.59 | 1.205191595 | Negative | Negative |
| CVD01619 | 3162 | 819.2 | 81.67490729 | 1:320 | Negative |
| CVD01620 | 3361 | 2040 | 90.2657602 | ≥1:640 | 1:80 |
| CVD01630 | 6073 | 2372 | 94.56118665 | ≥1:640 | 1:40 |
| CVD01701 | 7626 | 2130 | 95.46147979 | 1:320 | 1:40 |
| CVD01730 | 7940 | 4356 | 95.95728452 | ≥1:640 | 1:80 |
| CVD01734 | 31211 | 6239 | 96.18611747 | ≥1:640 | ≥1:640 |
| CVD01738 | 6638 | 1091 | 91.1136537 | ≥1:640 | 1:160 |
| CVD01741 | 2177 | 897.5 | 86.42257818 | ≥1:640 | 1:80 |
| CVD01763 | 1952 | 1827 | 92.14340198 | 1:160 | 1:20 |
| CVD01769 | 9894 | 2686 | 96.18611747 | ≥1:640 | 1:160 |
| CVD01776 | 281 | 87.09 | 2.860411899 | Negative | Negative |
| CVD01820 | 3042 | 1396 | 94.66056445 | 1:160 | 1:80 |
| CVD01823 | 300 | 199.4 | 58.96262395 | 1:40 | Negative |
| CVD01827 | 2731 | 597.6 | 77.49809306 | ≥1:640 | 1:160 |
| CVD01828 | 5930 | 1531 | 93.32570557 | 1:20 | 1:20 |
| CVD01852 | 429 | 107.1 | 68.30663616 | 1:320 | 1:20 |
| CVD01860 | 1368 | 859.7 | 55.98779558 | ≥1:640 | 1:20 |
| CVD01881 | 579 | 256.5 | 41.71632896 | 1:20 | Negative |
| CVD01920 | 677 | 411.9 | 89.35240366 | ≥1:640 | 1:40 |
| CVD01923 | 2064 | 875 | 86.92888359 | ≥1:640 | Negative |
| CVD01941 | 4872 | 956.9 | 81.3667064 | ≥1:640 | 1:320 |
| CVD01952 | 5187 | 1400 | 91.89511323 | ≥1:640 | 1:160 |
| CVD01953 | 8751 | 2653 | 90.9018673 | ≥1:640 | 1:160 |
| CVD01981 | 7271 | 3077 | 84.98212157 | ≥1:640 | 1:40 |
| CVD01982 | 10084 | 6458 | 94.99404052 | ≥1:640 | 1:320 |
| CVD02004 | 855 | 720.4 | 82.87644021 | 1:80 | Negative |
| CVD02007 | 2876 | 489.3 | 85.93563766 | 1:160 | 1:40 |
| CVD02016 | 9598 | 2169 | 90.66348828 | 1:320 | 1:160 |
| CVD02049 | 3872 | 1507 | 90.24390244 | ≥1:640 | 1:80 |
| CVD02052 | 2673 | 1478 | 91.07674004 | ≥1:640 | 1:20 |
| CVD02054 | 224 | 173.4 | 8.774538965 | Negative | Negative |
| CVD02059 | 669 | 1264 | 84.88994646 | 1:80 | 1:20 |
| CVD02060 | 737 | 482.5 | 71.59428911 | 1:20 | Negative |
| CVD02064 | 19837 | 3660 | 83.64069007 | ≥1:640 | 1:40 |
| CVD02066 | 201 | 169.3 | 4.074955384 | Negative | Negative |
| CVD02076 | 464 | 910.8 | 53.62879239 | 1:20 | Negative |
| CVD02078 | 1922 | 1944 | 90.92801904 | 1:320 | 1:80 |
| CVD02080 | 4014 | 3392 | 97.14455681 | ≥1:640 | 1:160 |
| CVD02082 | 4421 | 2011 | 90.27364664 | ≥1:640 | 1:80 |
| CVD02086 | 2866 | 1065 | 81.55859607 | 1:160 | 1:40 |
| CVD02170 | 4488 | 1823 | 61.37532134 | ≥1:640 | 1:80 |

### Example 2

Although only IgG antibody testing has been described in this Example 1, the principles of the tNLuc assay of the present disclosure should also be applicable to the detection of antibodies of other immunoglobulin isotypes such as IgM and IgA. This might be performed by using their binders as sensor proteins, for example single chain antibodies, nanobodies, or similar molecules specifically recognizing these isotypes. Additionally, the tNLuc strategy employed in the assays of the present disclosure has can be adapted for use in alternative diagnostic approaches to help detect responses to other pathogens and viruses such as SARS, MERS, and/or influenza.

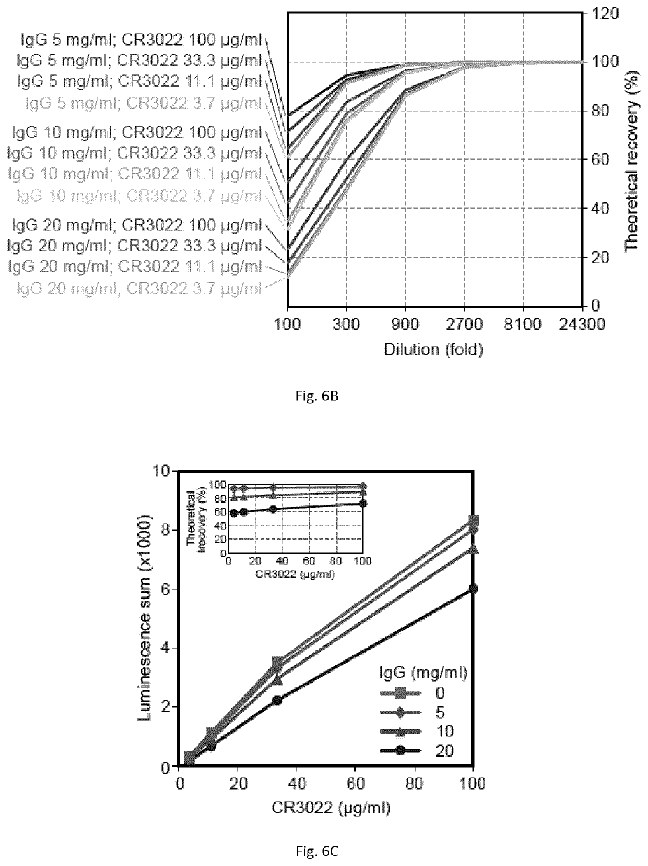

As illustrated in FIG. 7A, in contrast to IgG detection, a general IgM probe is used to bind IgM molecules. The general IgM probe can be any IgM binder conjugated, in the case of FIG. 7A, to β9 tag. We use a single domain antibody developed by NanoTag Biotechnologies (clone #2F2). Anti-S protein IgM levels in the sera of COVID-19 patients were evaluated using TNLuc-IgM assay of the present disclosure as described in Example 1. The results were plotted against days of symptom onset and the results are shown in FIG. 7B.

```
Sequence Listing
Δ11S nucleotide sequence
(SEQ ID NO: 1)
      1 ATGggccatc atcatcatca tcatcatcat ATGgttttta cacttgaaga ttttgtgggt
    61 gattgggaac aaactgctgc atataattta gatcaagttt tagaacaggg tggagtgagt
  121 tctcttttac aaaatcttgc agtctcagtt acaccaatac aaagaatagt tagaagtgga
  181 gaaaatgcat taaagatcga tatacatgta ataattcctt atgagggact atcagcagac
  241 caaatggcac aaattgagga agtattcaaa gttgtatatc cagtagacga tcatcacttt
  301 aaagtaatat taccttatgg aactttagta attgatggtg taacaccaaa tatgttaaat
  361 tattttggta gaccatacga agggattgca gtttttgatg gaaagaaaat aaccgtaaca
  421 ggcactttgt ggaatggaaa taaaataata gatgaaaggt taattacacc tgatTAA
Δ11S amino acid sequence
(SEQ ID NO: 2)
      1 MetGlyHisHisHisHisHisHisHisHisMetValPheThrLeuGluAspPheValGly
    21 AspTrpGluGlnThrAlaAlaTyrAsnLeuAspGlnValLeuGluGlnGlyGlyValSer
    41 SerLeuLeuGlnAsnLeuAlaValSerValThrProIleGInArgIleValArgSerGly
    61 GluAsnAlaLeuLysIleAspIleHisValIleIleProTyrGluGlyLeuSerAlaAsp
    81 GlnMetAlaGlnIleGluGluValPheLysValValTyrProValAspAspHisHisPhe
  101 LysValIleLeuProTyrGlyThrLeuValIleAspGlyValThrProAsnMetLeuAsn
  121 TyrPheGlyArgProTyrGluGlyIleAlaValPheAspGlyLysLysIleThrValThr
  141 GlyThrLeuTrpAsnGlyAsnLysIleIleAspGluArgLeuIleThrProAsp
β9 nucleotide sequence
(SEQ ID NO: 3)
      1 GGCTCCATGC TGTTCCGAGT AACCATCAAC AGT
β9 amino acid sequence
(SEQ ID NO: 4)
      1 GlySerMetLeuPheArgValThrIleAsnSer
β10 nucleotide sequence
(SEQ ID NO: 5)
      1 GTGAGCGGCT GGCGGCTGTT CAAGAAGATT AGC
β10 amino acid sequence
(SEQ ID NO: 6)
      1 ValSerGlyTrpArgLeuPheLysLys IleSer
β9-G nucleotide sequence
(SEQ ID NO: 7)
      1 ATGGGCTCCA TGCTGTTCCG AGTAACCATC AACAGTggag gtggatccGG TGGTGGAGGG
    61 AGCacctaca aacttGtcAT Taacggtaaa accctgaaag gtgaaaccac caccgaagct
  121 gttgacgctg ctaccgcgga aaaagttttc aaacagtacg ctaacgacaa cggtgttgac
  181 ggtgaatgga cctacgacga cgctaccaaa acctTcaccg taacggaaGG TGGCGGTAGC
  241 catcaccacc atcaccacTG A
β9-G amino acid sequence
(SEQ ID NO: 8)
      1 MetGlySerMetLeuPheArgValThrIleAsnSerGlyGlyGlySerGlyGlyGlyGly
    21 SerThrTyrLysLeuValIleAsnGlyLysThrLeuLysGlyGluThrThrThrGluAla
    41 ValAspAlaAlaThrAlaGluLysVal PheLysGlnTyrAlaAsnAspAsnGlyValAsp
    61 GlyGluTrpThrTyrAspAspAlaThrLysThrPheThrValThrGluGlyGlyGlySer
    81 HisHisHisHisHisHis
G-β9 nucleotide sequence
(SEQ ID NO: 9)
      1 ATGggccatc atcatcatca tcatGGTGGA GGGAGCacct acaaacttGt cATTaacggt
    61 aaaaccctga aaggtgaaac caccaccgaa gctgttgacg ctgctaccgc ggaaaaagtt
  121 ttcaaacagt acgctaacga caacggtgtt gacggtgaat ggacctacga cgacgctacc
  181 aaaacctTca ccgtaacgga aGGTGGCGGT AGCggtggcg gaggctctGG CTCCATGCTG
  241 TTCCGAGTAA CCATCAACAG Ttaa
G-β9 amino acid sequence
(SEQ ID NO: 10)
      1 MetGlyHisHisHisHisHisHisGlyGlyGlySerThrTyrLysLeuValIleAsnGly
    21 LysThrLeuLysGlyGluThrThrThrGluAlaValAspAlaAlaThrAlaGluLysVal
    41 PheLysGlnTyrAlaAsnAspAsnGlyValAspGlyGluTrpThrTyrAspAspAlaThr
    61 LysThrPheThrValThrGluGlyGlyGlySerGlyGlyGlyGlySerGlySerMetLeu
    81 PheArgValThrIleAsnSer
β9-G-39 nucleotide sequence
(SEQ ID NO: 11)
      1 ATGGGCTCCA TGCTGTTCCG AGTAACCATC AACAGTggag gtggatccGG TGGTGGAGGG
    61 AGCacctaca aacttGtcAT Taacggtaaa accctgaaag gtgaaaccac caccgaagct
  121 gttgacgctg ctaccgcgga aaaagttttc aaacagtacg ctaacgacaa cggtgttgac
  181 ggtgaatgga cctacgacga cgctaccaaa acctTcaccg taacggaaGG AGGAGGATCT
  241 GGAGGTGGAT CCGGCTCCAT GCTGTTCCGA GTAACCATCA ACAGTGGCGG AAGCCATCAT
  301 CACCACCATC ATCACCATTG A
β9-G-39 amino acid sequence
(SEQ ID NO: 12)
      1 MetGlySerMetLeuPheArgValThrIleAsnSerGlyGlyGlySerGlyGlyGlyGly
    21 SerThrTyrLysLeuValIleAsnGlyLysThrLeuLysGlyGluThrThrThrGluAla
    41 ValAspAlaAlaThrAlaGluLysValPheLysGlnTyrAlaAsnAspAsnGlyValAsp
    61 GlyGluTrpThrTyrAspAspAlaThrLysThr PheThrValThrGluGlyGlyGlySer
    81 GlyGlyGlySerGlySerMetLeuPheArgValThrIleAsnSerGlyGlySerHisHis
  101 HisHisHisHisHisHis
β10-RBD nucleotide sequence
(SEQ ID NO: 13)
      1 ATGCGGCTTC CGGGTGCGAT GCCAGCTCTG GCCCTCAAAG GCGAGCTGCT GTTGCTGTCT
    61 CTCCTGTTAC TTCTGGAACC ACAGATCTCT CAGGGCGGTG GaGTGAGCGG CTGGCGGCTG
  121 TTCAAGAAGA TTAGCGGTGG CGGAGGATCT GGAGGTGGAT CCagggtgca gccaaccgag
  181 tctatcgtgc gctttcctaa tatcacaaac ctgtgcccat ttggcgaggt gttcaacgca
  241 acccgcttcg ccagcgtgta cgcctggaat aggaagcgga tcagcaactg cgtggccgac
  301 tatagcgtgc tgtacaactc cgcctctttc agcaccttta agtgctatgg cgtgtccccc
  361 acaaagctga atgacctgtg ctttaccaac gtctacgccg attctttcgt gatcaggggc
  421 gacgaggtgc gccagatcgc ccccggccag acaggcaaga tcgcagacta caattataag
  481 ctgccagacg atttcaccgg ctgcgtgatc gcctggaaca gcaacaatct ggattccaaa
  541 gtgggcggca actacaatta tctgtaccgg ctgtttagaa agagcaatct gaagcccttc
  601 gagagggaca tctctacaga aatctaccag gccggcagca ccccttgcaa tggcgtggag
  661 ggctttaact gttatttccc actccagtcc tacggcttcc agcccacaaa cggcgtgggc
  721 tatcagcctt accgcgtggt ggtgctgagc tttgagctgc tgcacgcccc agcaacagtg
  781 tgcggcccca agaagtccac caatctggtg aagaacaagt gcgtgaactt cGGCGGAAGC
  841 CATCATCACC ACCATCATCA CCATTGA
β10-RBD amino acid sequence
(SEQ ID NO: 14)
      1 MetArgLeuProGlyAlaMetProAlaLeuAlaLeuLysGlyGluLeuLeuLeuLeuSer
    21 LeuLeuLeuLeuLeuGluProGlnIleSerGlnGlyGlyGlyValSerGlyTrpArgLeu
    41 PheLysLysIleSerGlyGlyGlyGlySerGlyGlyGlySerArgValGlnProThrGlu
    61 SerIleValArgPheProAsnIleThrAsnLeuCysProPheGlyGluValPheAsnAla
    81 ThrArgPheAlaSerValTyrAlaTrpAsnArgLysArgIleSerAsnCysValAlaAsp
  101 TyrSerValLeuTyrAsnSerAlaSerPheSerThrPheLysCysTyrGlyValSerPro
  121 ThrLysLeuAsnAspLeuCysPheThrAsnValTyrAlaAspSerPheValIleArgGly
  141 AspGluValArgGlnIleAlaProGlyGlnThrGlyLysIleAlaAspTyrAsnTyrLys
  161 LeuProAspAspPheThrGlyCysValIleAlaTrpAsnSerAsnAsnLeuAspSerLys
  181 ValGlyGlyAsnTyrAsnTyrLeuTyrArgLeuPheArgLysSerAsnLeuLysProPhe
  201 GluArgAspIleSerThrGluIleTyrGlnAlaGlySerThrProCysAsnGlyValGlu
  221 GlyPheAsnCysTyrPheProLeuGlnSerTyrGlyPheGlnProThrAsnGlyValGly
  241 TyrGlnProTyrArgValValValLeuSerPheGluLeuLeuHisAlaProAlaThrVal
  261 CysGlyProLysLysSerThrAsnLeuValLysAsnLysCysValAsnPheGlyGlySer
  281 HisHisHisHisHisHisHisHis
RBD-β10 nucleotide sequence
(SEQ ID NO: 15)
      1 atgttcgtct tcctggtcct gctgcctctg gtctcctcac agagggtgca gccaaccgag
    61 tctatcgtgc gctttcctaa tatcacaaac ctgtgcccat ttggcgaggt gttcaacgca
  121 acccgcttcg ccagcgtgta cgcctggaat aggaagcgga tcagcaactg cgtggccgac
  181 tatagcgtgc tgtacaactc cgcctctttc agcaccttta agtgctatgg cgtgtccccc
  241 acaaagctga atgacctgtg ctttaccaac gtctacgccg attctttcgt gatcaggggc
  301 gacgaggtgc gccagatcgc ccccggccag acaggcaaga tcgcagacta caattataag
  361 ctgccagacg atttcaccgg ctgcgtgatc gcctggaaca gcaacaatct ggattccaaa
  421 gtgggcggca actacaatta tctgtaccgg ctgtttagaa agagcaatct gaagcccttc
  481 gagagggaca tctctacaga aatctaccag gccggcagca ccccttgcaa tggcgtggag
  541 ggctttaact gttatttccc actccagtcc tacggcttcc agcccacaaa cggcgtgggc
  601 tatcagcctt accgcgtggt ggtgctgagc tttgagctgc tgcacgcccc agcaacagtg
  661 tgcggcccca agaagtccac caatctggtg aagaacaagt gcgtgaactt cGGCGGAGGA
  721 GGATCTGGAG GTGGATCCGT GAGCGGCTGG CGGCTGTTCA AGAAGATTAG CGGCGGAAGC
  781 CATCATCACC ACCATCATCA CCATTGA
RBD-β10 amino acid sequence
(SEQ ID NO: 16)
      1 MetPheValPheLeuValLeuLeuProLeuValSerSerGlnArgValGlnProThrGlu
    21 SerIleValArgPheProAsnIleThrAsnLeuCysProPheGlyGluValPheAsnAla
    41 ThrArgPheAlaSerValTyrAlaTrpAsnArgLysArgIleSerAsnCysValAlaAsp
    61 TyrSerValLeuTyrAsnSerAlaSerPheSerThrPheLysCysTyrGlyValSerPro
    81 ThrLysLeuAsnAspLeuCysPheThrAsnValTyrAlaAspSerPheValIleArgGly
  101 AspGluValArgGlnIleAlaProGlyGlnThrGlyLysIleAlaAspTyrAsnTyrLys
  121 LeuProAspAspPheThrGlyCysValIleAlaTrpAsnSerAsnAsnLeuAspSerLys
  141 ValGlyGlyAsnTyrAsnTyrLeuTyrArgLeuPheArgLysSerAsnLeuLysProPhe
  161 GluArgAspIleSerThrGluIleTyrGlnAlaGlySerThrProCysAsnGlyValGlu
  181 GlyPheAsnCysTyrPheProLeuGlnSerTyrGlyPheGlnProThrAsnGlyValGly
  201 TyrGlnProTyrArgValValValLeuSerPheGluLeuLeuHisAlaProAlaThrVal
  221 CysGlyProLysLysSerThrAsnLeuValLysAsnLysCysValAsnPheGlyGlyGly
  241 GlySerGlyGlyGlySerValSerGlyTrpArgLeuPheLysLysIleSerGlyGlySer
  261 HisHisHisHisHisHisHisHis
β10-RBD-β10 nucleotide sequence
(SEQ ID NO: 17)
      1 ATGCGGCTTC CGGGTGCGAT GCCAGCTCTG GCCCTCAAAG GCGAGCTGCT GTTGCTGTCT
    61 CTCCTGTTAC TTCTGGAACC ACAGATCTCT CAGGGCGGTG GaGTGAGCGG CTGGCGGCTG
  121 TTCAAGAAGA TTAGCGGTGG CGGAGGATCT GGAGGTGGAT CCagggtgca gccaaccgag
  181 tctatcgtgc gctttcctaa tatcacaaac ctgtgcccat ttggcgaggt gttcaacgca
  241 acccgcttcg ccagcgtgta cgcctggaat aggaagcgga tcagcaactg cgtggccgac
  301 tatagcgtgc tgtacaactc cgcctctttc agcaccttta agtgctatgg cgtgtccccc
  361 acaaagctga atgacctgtg ctttaccaac gtctacgccg attctttcgt gatcaggggc
  421 gacgaggtgc gccagatcgc ccccggccag acaggcaaga tcgcagacta caattataag
  481 ctgccagacg atttcaccgg ctgcgtgatc gcctggaaca gcaacaatct ggattccaaa
  541 gtgggcggca actacaatta tctgtaccgg ctgtttagaa agagcaatct gaagcccttc
  601 gagagggaca tctctacaga aatctaccag gccggcagca ccccttgcaa tggcgtggag
  661 ggctttaact gttatttccc actccagtcc tacggcttcc agcccacaaa cggcgtgggc
  721 tatcagcctt accgcgtggt ggtgctgagc tttgagctgc tgcacgcccc agcaacagtg
  781 tgcggcccca agaagtccac caatctggtg aagaacaagt gcgtgaactt cGGCGGAGGA
  841 GGATCTGGAG GTGGATCCGT GAGCGGCTGG CGGCTGTTCA AGAAGATTAG CGGCGGAAGC
  901 CATCATCACC ACCATCATCA CCATTGA
β10-RBD-β10 amino acid sequence
(SEQ ID NO: 18)
      1 MetArgLeuProGlyAlaMetProAlaLeuAlaLeuLysGlyGluLeuLeuLeuLeuSer
    21 LeuLeuLeuLeuLeuGluProGlnIleSerGlnGlyGlyGlyValSerGlyTrpArgLeu
    41 PheLysLysIleSerGlyGlyGlyGlySerGlyGlyGlySerArgValGlnProThrGlu
    61 SerIleValArgPheProAsnIleThrAsnLeuCysProPheGlyGluValPheAsnAla
    81 ThrArgPheAlaSerValTyrAlaTrpAsnArgLysArgIleSerAsnCysValAlaAsp
  101 TyrSerValLeuTyrAsnSerAlaSerPheSerThrPheLysCysTyrGlyValSerPro
  121 ThrLysLeuAsnAspLeuCysPheThrAsnValTyrAlaAspSerPheValIleArgGly
  141 AspGluValArgGlnIleAlaProGlyGlnThrGlyLysIleAlaAspTyrAsnTyrLys
  161 LeuProAspAspPheThrGlyCysValIleAlaTrpAsnSerAsnAsnLeuAspSerLys
  181 ValGlyGlyAsnTyrAsnTyrLeuTyrArgLeuPheArgLysSerAsnLeuLysProPhe
  201 GluArgAspIleSerThrGluIleTyrGlnAlaGlySerThrProCysAsnGlyValGlu
  221 GlyPheAsnCysTyrPheProLeuGlnSerTyrGlyPheGlnProThrAsnGlyValGly
  241 TyrGlnProTyrArgValValValLeuSerPheGluLeuLeuHisAlaProAlaThrVal
  261 CysGlyProLysLysSerThrAsnLeuValLysAsnLysCysValAsnPheGlyGlyGly
  281 GlySerGlyGlyGlySerValSerGlyTrpArgLeuPheLysLysIleSerGlyGlySer
  301 HisHisHisHisHisHisHisHis
β10-S nucleotide sequence
(SEQ ID NO: 19)
      1 ATGCGGCTTC CGGGTGCGAT GCCAGCTCTG GCCCTCAAAG GCGAGCTGCT GTTGCTGTCT
    61 CTCCTGTTAC TTCTGGAACC ACAGATCTCT CAGGGCGGTG GaGTGAGCGG CTGGCGGCTG
  121 TTCAAGAAGA TTAGCGGTGG CGGAGGATCT GGAGGTGGAT CCtgcgtcaa tctgacaact
  181 cggactcagc tgccacctgc ttatactaat agcttcacca gaggcgtgta ctatcctgac
  241 aaggtgttta gaagctccgt gctgcactct acacaggatc tgtttctgcc attctttagc
  301 aacgtgacct ggttccacgc catccacgtg agcggcacca atggcacaaa gcggttcgac
  361 aatcccgtgc tgccttttaa cgatggcgtg tacttcgcct ctaccgagaa gagcaacatc
  421 atcagaggct ggatctttgg caccacactg gactccaaga cacagtctct gctgatcgtg
  481 aacaatgcca ccaacgtggt catcaaggtg tgcgagttcc agttttgtaa tgatcccttc
  541 ctgggcgtgt actatcacaa gaacaataag agctggatgg agtccgagtt tagagtgtat
  601 tctagcgcca acaactgcac atttgagtac gtgagccagc ctttcctgat ggacctggag
  661 ggcaagcagg gcaatttcaa gaacctgagg gagttcgtgt ttaagaatat cgacggctac
  721 ttcaaaatct actctaagca cacccccatc aacctggtgc gcgacctgcc tcagggcttc
  781 agcgccctgg agcccctggt ggatctgcct atcggcatca acatcacccg gtttcagaca
  841 ctgctggccc tgcacagaag ctacctgaca cccggcgact cctctagcgg atggaccgcc
  901 ggcgctgccg cctactatgt gggctacctc cagccccgga ccttcctgct gaagtacaac
  961 gagaatggca ccatcacaga cgcagtggat tgcgccctgg accccctgag cgagacaaag
1021 tgtacactga agtcctttac cgtggagaag ggcatctatc agacatccaa tttcagggtg
1081 cagccaaccg agtctatcgt gcgctttcct aatatcacaa acctgtgccc atttggcgag
1141 gtgttcaacg caacccgctt cgccagcgtg tacgcctgga ataggaagcg gatcagcaac
1201 tgcgtggccg actatagcgt gctgtacaac tccgcctctt tcagcacctt taagtgctat
1261 ggcgtgtccc ccacaaagct gaatgacctg tgctttacca acgtctacgc cgattctttc
1321 gtgatcaggg gcgacgaggt gcgccagatc gcccccggcc agacaggcaa gatcgcagac
1381 tacaattata agctgccaga cgatttcacc ggctgcgtga tcgcctggaa cagcaacaat
1441 ctggattcca aagtgggcgg caactacaat tatctgtacc ggctgtttag aaagagcaat
1501 ctgaagccct tcgagaggga catctctaca gaaatctacc aggccggcag caccccttgc
1561 aatggcgtgg agggctttaa ctgttatttc ccactccagt cctacggctt ccagcccaca
1621 aacggcgtgg gctatcagcc ttaccgcgtg gtggtgctga gctttgagct gctgcacgcc
1681 ccagcaacag tgtgcggccc caagaagtcc accaatctgg tgaagaacaa gtgcgtgaac
1741 ttcaacttca acggcctgac cggcacaggc gtgctgaccg agtccaacaa gaagttcctg
1801 ccatttcagc agttcggcag ggacatcgca gataccacag acgccgtgcg cgacccacag
1861 accctggaga tcctggacat cacaccctgc tctttcggcg gcgtgagcgt gatcacaccc
1921 ggcaccaata caagcaacca ggtggccgtg ctgtatcagg acgtgaattg taccgaggtg
1981 cccgtggcta tccacgccga tcagctgacc ccaacatggc gggtgtacag caccggctcc
2041 aacgtcttcc agacaagagc cggatgcctg atcggagcag agcacgtgaa caattcctat
2101 gagtgcgaca tcccaatcgg cgccggcatc tgtgcctctt accagaccca gacaaactct
2161 cccGgaagCg ccAgCagcgt ggcctcccag tctatcatcg cctataccat gtccctgggc
2221 gccgagaaca gcgtggccta ctctaacaat agcatcgcca tcccaaccaa cttcacaatc
2281 tctgtgacca cagagatcct gcccgtgtcc atgaccaaga catctgtgga ctgcacaatg
2341 tatatctgtg gcgattctac cgagtgcagc aacctgctgc tccagtacgg cagcttttgt
2401 acccagctga atagagccct gacaggcatc gccgtggagc aggataagaa cacacaggag
2461 gtgttcgccc aggtgaagca aatctacaag acccccccta tcaaggactt tggcggcttc
2521 aatttttccc agatcctgcc tgatccatcc aagccttcta agcggagctt tatcgaggac
2581 ctgctgttca acaaggtgac cctggccgat gccggcttca tcaagcagta tggcgattgc
2641 ctgggcgaca tcgcagccag ggacctgatc tgcgcccaga agtttaatgg cctgaccgtg
2701 ctgccacccc tgctgacaga tgagatgatc gcacagtaca caagcgccct gctggccggc
2761 accatcacat ccggatggac cttcggcgca ggagccgccc tccagatccc ctttgccatg
2821 cagatggcct ataggttcaa cggcatcggc gtgacccaga atgtgctgta cgagaaccag
2881 aagctgatcg ccaatcagtt taactccgcc atcggcaaga tccaggacag cctgtcctct
2941 acagccagcg ccctgggcaa gctccaggat gtggtgaatc agaacgccca ggccctgaat
3001 accctggtga agcagctgag cagcaacttc ggcgccatct ctagcgtgct gaatgacatc
3061 ctgagccggc tggacCCTCC Tgaggcagag gtgcagatcg accggctgat caccggccgg
3121 ctccagagcc tccagaccta tgtgacacag cagctgatca gggccgccga gatcagggcc
3181 agcgccaatc tggcagcaac caagatgtcc gagtgcgtgc tgggccagtc taagagagtg
3241 gacttttgtg gcaagggcta tcacctgatg tccttccctc agtctgcccc acacggcgtg
3301 gtgtttctgc acgtgaccta cgtgcccgcc caggagaaga acttcaccac agcccctgcc
3361 atctgccacg atggcaaggc ccactttcca agggagggcg tgttcgtgtc caacggcacc
3421 cactggtttg tgacacagcg caatttctac gagccccaga tcatcaccac agacaacacc
3481 ttcgtgagcg gcaactgtga cgtggtcatc ggcatcgtga acaataccgt gtatgatcca
3541 ctccagcccg agctggacag ctttaaggag gagctggata agtatttcaa gaatcacacc
3601 tcccctgacg tggatctggg cgacatcagc ggcatcaatg cctccgtggt gaacatccag
3661 aaggagatcg accgcctgaa cgaggtggct aagaatctga acgagagcct gatcgacctc
3721 caggagctgg gcaagtatga gcagtacatc aagtggcccG GaGGCAGCGG aGGCTACATC
3781 CCCGAGGCCC CtCGCGAtGG aCAGGCtTAC GTGCGCAAGG ACGGCGAGTG GGTGCTGCTG
3841 AGCACCTTCC TGGGCGGAAG CCATCATCAC CACCATCATC ACCATTGA
β10-S amino acid sequence
(SEQ ID NO: 20)
      1 MetArgLeuProGlyAlaMetProAlaLeuAlaLeuLysGlyGluLeuLeuLeuLeuSer
    21 LeuLeuLeuLeuLeuGluProGlnIleSerGlnGlyGlyGlyValSerGlyTrpArgLeu
    41 PheLysLysIleSerGlyGlyGlyGlySerGlyGlyGlySerCysValAsnLeuThrThr
    61 ArgThrGlnLeuProProAlaTyrThrAsnSerPheThrArgGlyValTyrTyrProAsp
    81 LysValPheArgSerSerValLeuHisSerThrGlnAspLeuPheLeuProPhePheSer
  101 AsnValThrTrpPheHisAlaIleHisValSerGlyThrAsnGlyThrLysArgPheAsp
  121 AsnProValLeuProPheAsnAspGlyValTyrPheAlaSerThrGluLysSerAsnIle
  141 IleArgGlyTrpIlePheGlyThrThrLeuAspSerLysThrGlnSerLeuLeuIleVal
  161 AsnAsnAlaThrAsnValValIleLysValCysGluPheGInPheCysAsnAspProPhe
  181 LeuGlyValTyrTyrHisLysAsnAsnLysSerTrpMetGluSerGluPheArgValTyr
  201 SerSerAlaAsnAsnCysThrPheGluTyrValSerGlnProPheLeuMetAspLeuGlu
  221 GlyLysGlnGlyAsnPheLysAsnLeuArgGluPheValPheLysAsnIleAspGlyTyr
  241 PheLysIleTyrSerLysHisThrProIleAsnLeuValArgAspLeuProGInGlyPhe
  261 SerAlaLeuGluProLeuValAspLeuProIleGlyIleAsnIleThrArgPheGlnThr
  281 LeuLeuAlaLeuHisArgSerTyrLeuThrProGlyAspSerSerSerGlyTrpThrAla
  301 GlyAlaAlaAlaTyrTyrValGlyTyrLeuGlnProArgThrPheLeuLeuLysTyrAsn
  321 GluAsnGlyThrIleThrAspAlaValAspCysAlaLeuAspProLeuSerGluThrLys
  341 CysThrLeuLysSerPheThrValGluLysGlyIleTyrGlnThrSerAsnPheArgVal
  361 GlnProThrGluSerIleValArgPheProAsnIleThrAsnLeuCysProPheGlyGlu
  381 ValPheAsnAlaThrArgPheAlaSerValTyrAlaTrpAsnArgLysArgIleSerAsn
  401 CysValAlaAspTyrSerValLeuTyrAsnSerAlaSerPheSerThrPheLysCysTyr
  421 GlyValSerProThrLysLeuAsnAspLeuCysPheThrAsnValTyrAlaAspSerPhe
  441 ValIleArgGlyAspGluValArgGlnIleAlaProGlyGlnThrGlyLysIleAlaAsp
  461 TyrAsnTyrLysLeuProAspAspPheThrGlyCysValIleAlaTrpAsnSerAsnAsn
  481 LeuAspSerLysValGlyGlyAsnTyrAsnTyrLeuTyrArgLeuPheArgLysSerAsn
  501 LeuLysProPheGluArgAspIleSerThrGluIleTyrGlnAlaGlySerThrProCys
  521 AsnGlyValGluGlyPheAsnCysTyrPheProLeuGlnSerTyrGlyPheGlnProThr
  541 AsnGlyValGlyTyrGlnProTyrArgValValValLeuSerPheGluLeuLeuHisAla
  561 ProAlaThrValCysGlyProLysLysSerThrAsnLeuValLysAsnLysCysValAsn
  581 PheAsnPheAsnGlyLeuThrGlyThrGlyValLeuThrGluSerAsnLysLysPheLeu
  601 ProPheGlnGlnPheGlyArgAspIleAlaAspThrThrAspAlaValArgAspProGln
  621 ThrLeuGluIleLeuAspIleThrProCysSerPheGlyGlyValSerValIleThrPro
  641 GlyThrAsnThrSerAsnGlnValAlaValLeuTyrGlnAspValAsnCysThrGluVal
  661 ProValAlaIleHisAlaAspGlnLeuThrProThrTrpArgValTyrSerThrGlySer
  681 AsnValPheGlnThrArgAlaGlyCysLeuIleGlyAlaGluHisValAsnAsnSerTyr
  701 GluCysAspIleProIleGlyAlaGlyIleCysAlaSerTyrGlnThrGlnThrAsnSer
  721 ProGlySerAlaSerSerValAlaSerGlnSerIleIleAlaTyrThrMetSerLeuGly
  741 AlaGluAsnSerValAlaTyrSerAsnAsnSerIleAlaIleProThrAsnPheThrIle
  761 SerValThrThrGluIleLeuProValSerMetThrLysThrSerValAspCysThrMet
  781 TyrIleCysGlyAspSerThrGluCysSerAsnLeuLeuLeuGlnTyrGlySerPheCys
  801 ThrGlnLeuAsnArgAlaLeuThrGlyIleAlaValGluGInAspLysAsnThrGlnGlu
  821 ValPheAlaGlnValLysGlnIleTyrLysThrProProIleLysAspPheGlyGlyPhe
  841 AsnPheSerGlnIleLeuProAspProSerLysProSerLysArgSerPheIleGluAsp
  861 LeuLeuPheAsnLysValThrLeuAlaAspAlaGlyPheIleLysGlnTyrGlyAspCys
  881 LeuGlyAspIleAlaAlaArgAspLeuIleCysAlaGInLysPheAsnGlyLeuThrVal
  901 LeuProProLeuLeuThrAspGluMetIleAlaGlnTyrThrSerAlaLeuLeuAlaGly
  921 ThrIleThrSerGlyTrpThrPheGlyAlaGlyAlaAlaLeuGlnIleProPheAlaMet
  941 GlnMetAlaTyrArgPheAsnGlyIleGlyValThrGlnAsnValLeuTyrGluAsnGln
  961 LysLeuIleAlaAsnGlnPheAsnSerAlaIleGlyLysIleGInAspSerLeuSerSer
  981 ThrAlaSerAlaLeuGlyLysLeuGlnAspValValAsnGlnAsnAlaGInAlaLeuAsn
1001 ThrLeuValLysGlnLeuSerSerAsnPheGlyAlaIleSerSerValLeuAsnAspIle
1021 LeuSerArgLeuAspProProGluAlaGluValGlnIleAspArgLeuIleThrGlyArg
1041 LeuGlnSerLeuGlnThrTyrValThrGlnGlnLeuIleArgAlaAlaGluIleArgAla
1061 SerAlaAsnLeuAlaAlaThrLysMetSerGluCysValLeuGlyGlnSerLysArgVal
1081 AspPheCysGlyLysGlyTyrHisLeuMetSerPheProGlnSerAlaProHisGlyVal
1101 ValPheLeuHisValThrTyrValProAlaGInGluLysAsnPheThrThrAlaProAla
1121 IleCysHisAspGlyLysAlaHisPheProArgGluGlyValPheValSerAsnGlyThr
1141 HisTrpPheValThrGlnArgAsnPheTyrGluProGlnIleIleThrThrAspAsnThr
1161 PheValSerGlyAsnCysAspValValIleGlyIleValAsnAsnThrValTyrAspPro
1181 LeuGlnProGluLeuAspSerPheLysGluGluLeuAspLysTyrPheLysAsnHisThr
1201 SerProAspValAspLeuGlyAspIleSerGlyIleAsnAlaSerValValAsnIleGln
1221 LysGluIleAspArgLeuAsnGluValAlaLysAsnLeuAsnGluSerLeuIleAspLeu
1241 GlnGluLeuGlyLysTyrGluGlnTyrIleLysTrpProGlyGlySerGlyGlyTyrIle
1261 ProGluAlaProArgAspGlyGlnAlaTyrValArgLysAspGlyGluTrpValLeuLeu
1281 SerThrPheLeuGlyGlySerHisHisHisHisHisHisHisHis
S-β 10 nucleotide sequence
(SEQ ID NO: 21)
      1 atgttcgtct tcctggtcct gctgcctctg gtctcctcac agtgcgtcaa tctgacaact
    61 cggactcagc tgccacctgc ttatactaat agcttcacca gaggcgtgta ctatcctgac
  121 aaggtgttta gaagctccgt gctgcactct acacaggatc tgtttctgcc attctttagc
  181 aacgtgacct ggttccacgc catccacgtg agcggcacca atggcacaaa gcggttcgac
  241 aatcccgtgc tgccttttaa cgatggcgtg tacttcgcct ctaccgagaa gagcaacatc
  301 atcagaggct ggatctttgg caccacactg gactccaaga cacagtctct gctgatcgtg
  361 aacaatgcca ccaacgtggt catcaaggtg tgcgagttcc agttttgtaa tgatcccttc
  421 ctgggcgtgt actatcacaa gaacaataag agctggatgg agtccgagtt tagagtgtat
  481 tctagcgcca acaactgcac atttgagtac gtgagccagc ctttcctgat ggacctggag
  541 ggcaagcagg gcaatttcaa gaacctgagg gagttcgtgt ttaagaatat cgacggctac
  601 ttcaaaatct actctaagca cacccccatc aacctggtgc gcgacctgcc tcagggcttc
  661 agcgccctgg agcccctggt ggatctgcct atcggcatca acatcacccg gtttcagaca
  721 ctgctggccc tgcacagaag ctacctgaca cccggcgact cctctagcgg atggaccgcc
  781 ggcgctgccg cctactatgt gggctacctc cagccccgga ccttcctgct gaagtacaac
  841 gagaatggca ccatcacaga cgcagtggat tgcgccctgg accccctgag cgagacaaag
  901 tgtacactga agtcctttac cgtggagaag ggcatctatc agacatccaa tttcagggtg
  961 cagccaaccg agtctatcgt gcgctttcct aatatcacaa acctgtgccc atttggcgag
1021 gtgttcaacg caacccgctt cgccagcgtg tacgcctgga ataggaagcg gatcagcaac
1081 tgcgtggccg actatagcgt gctgtacaac tccgcctctt tcagcacctt taagtgctat
1141 ggcgtgtccc ccacaaagct gaatgacctg tgctttacca acgtctacgc cgattctttc
1201 gtgatcaggg gcgacgaggt gcgccagatc gcccccggcc agacaggcaa gatcgcagac
1261 tacaattata agctgccaga cgatttcacc ggctgcgtga tcgcctggaa cagcaacaat
1321 ctggattcca aagtgggcgg caactacaat tatctgtacc ggctgtttag aaagagcaat
1381 ctgaagccct tcgagaggga catctctaca gaaatctacc aggccggcag caccccttgc
1441 aatggcgtgg agggctttaa ctgttatttc ccactccagt cctacggctt ccagcccaca
1501 aacggcgtgg gctatcagcc ttaccgcgtg gtggtgctga gctttgagct gctgcacgcc
1561 ccagcaacag tgtgcggccc caagaagtcc accaatctgg tgaagaacaa gtgcgtgaac
1621 ttcaacttca acggcctgac cggcacaggc gtgctgaccg agtccaacaa gaagttcctg
1681 ccatttcagc agttcggcag ggacatcgca gataccacag acgccgtgcg cgacccacag
1741 accctggaga tcctggacat cacaccctgc tctttcggcg gcgtgagcgt gatcacaccc
1801 ggcaccaata caagcaacca ggtggccgtg ctgtatcagg acgtgaattg taccgaggtg
1861 cccgtggcta tccacgccga tcagctgacc ccaacatggc gggtgtacag caccggctcc
1921 aacgtcttcc agacaagagc cggatgcctg atcggagcag agcacgtgaa caattcctat
1981 gagtgcgaca tcccaatcgg cgccggcatc tgtgcctctt accagaccca gacaaactct
2041 cccGgaagCg ccAgCagcgt ggcctcccag tctatcatcg cctataccat gtccctgggc
2101 gccgagaaca gcgtggccta ctctaacaat agcatcgcca tcccaaccaa cttcacaatc
2161 tctgtgacca cagagatcct gcccgtgtcc atgaccaaga catctgtgga ctgcacaatg
2221 tatatctgtg gcgattctac cgagtgcagc aacctgctgc tccagtacgg cagcttttgt
2281 acccagctga atagagccct gacaggcatc gccgtggagc aggataagaa cacacaggag
2341 gtgttcgccc aggtgaagca aatctacaag acccccccta tcaaggactt tggcggcttc
2401 aatttttccc agatcctgcc tgatccatcc aagccttcta agcggagctt tatcgaggac
2461 ctgctgttca acaaggtgac cctggccgat gccggcttca tcaagcagta tggcgattgc
2521 ctgggcgaca tcgcagccag ggacctgatc tgcgcccaga agtttaatgg cctgaccgtg
2581 ctgccacccc tgctgacaga tgagatgatc gcacagtaca caagcgccct gctggccggc
2641 accatcacat ccggatggac cttcggcgca ggagccgccc tccagatccc ctttgccatg
2701 cagatggcct ataggttcaa cggcatcggc gtgacccaga atgtgctgta cgagaaccag
2761 aagctgatcg ccaatcagtt taactccgcc atcggcaaga tccaggacag cctgtcctct
2821 acagccagcg ccctgggcaa gctccaggat gtggtgaatc agaacgccca ggccctgaat
2881 accctggtga agcagctgag cagcaacttc ggcgccatct ctagcgtgct gaatgacatc
2941 ctgagccggc tggacCCTCC Tgaggcagag gtgcagatcg accggctgat caccggccgg
3001 ctccagagcc tccagaccta tgtgacacag cagctgatca gggccgccga gatcagggcc
3061 agcgccaatc tggcagcaac caagatgtcc gagtgcgtgc tgggccagtc taagagagtg
3121 gacttttgtg gcaagggcta tcacctgatg tccttccctc agtctgcccc acacggcgtg
3181 gtgtttctgc acgtgaccta cgtgcccgcc caggagaaga acttcaccac agcccctgcc
3241 atctgccacg atggcaaggc ccactttcca agggagggcg tgttcgtgtc caacggcacc
3301 cactggtttg tgacacagcg caatttctac gagccccaga tcatcaccac agacaacacc
3361 ttcgtgagcg gcaactgtga cgtggtcatc ggcatcgtga acaataccgt gtatgatcca
3421 ctccagcccg agctggacag ctttaaggag gagctggata agtatttcaa gaatcacacc
3481 tcccctgacg tggatctggg cgacatcagc ggcatcaatg cctccgtggt gaacatccag
3541 aaggagatcg accgcctgaa cgaggtggct aagaatctga acgagagcct gatcgacctc
3601 caggagctgg gcaagtatga gcagtacatc aagtggcccG GaGGCAGCGG aGGCTACATC
3661 CCCGAGGCCC CtCGCGAtGG aCAGGCtTAC GTGCGCAAGG ACGGCGAGTG GGTGCTGCTG
3721 AGCACCTTCC TGGGCGGAGG AGGATCTGGA GGTGGATCCG TGAGCGGCTG GCGGCTGTTC
3781 AAGAAGATTA GCGGCGGAAG CCATCATCAC CACCATCATC ACCATTGA
S-β10 amino acid sequence
(SEQ ID NO: 22)
      1 MetPheValPheLeuValLeuLeuProLeuValSerSerGInCysValAsnLeuThrThr
    21 ArgThrGlnLeuProProAlaTyrThrAsnSerPheThrArgGlyValTyrTyrProAsp
    41 LysValPheArgSerSerValLeuHisSerThrGlnAspLeuPheLeuProPhePheSer
    61 AsnValThrTrpPheHisAlaIleHisValSerGlyThrAsnGlyThrLysArgPheAsp
    81 AsnProValLeuProPheAsnAspGlyValTyrPheAlaSerThrGluLysSerAsnIle
  101 IleArgGlyTrpIlePheGlyThrThrLeuAspSerLysThrGlnSerLeuLeuIleVal
  121 AsnAsnAlaThrAsnValValIleLysValCysGluPheGInPheCysAsnAspProPhe
  141 LeuGlyValTyrTyrHisLysAsnAsnLysSerTrpMetGluSerGluPheArgValTyr
  161 SerSerAlaAsnAsnCysThrPheGluTyrValSerGlnProPheLeuMetAspLeuGlu
  181 GlyLysGlnGlyAsnPheLysAsnLeuArgGluPheValPheLysAsnIleAspGlyTyr
  201 PheLysIleTyrSerLysHisThrProIleAsnLeuValArgAspLeuProGInGlyPhe
  221 SerAlaLeuGluProLeuValAspLeuProIleGlyIleAsnIleThrArgPheGlnThr
  241 LeuLeuAlaLeuHisArgSerTyrLeuThrProGlyAspSerSerSerGlyTrpThrAla
  261 GlyAlaAlaAlaTyrTyrValGlyTyrLeuGlnProArgThrPheLeuLeuLysTyrAsn
  281 GluAsnGlyThrIleThrAspAlaValAspCysAlaLeuAspProLeuSerGluThrLys
  301 CysThrLeuLysSerPheThrValGluLysGlyIleTyrGlnThrSerAsnPheArgVal
  321 GlnProThrGluSerIleValArgPheProAsnIleThrAsnLeuCysProPheGlyGlu
  341 ValPheAsnAlaThrArgPheAlaSerValTyrAlaTrpAsnArgLysArgIleSerAsn
  361 CysValAlaAspTyrSerValLeuTyrAsnSerAlaSerPheSerThrPheLysCysTyr
  381 GlyValSerProThrLysLeuAsnAspLeuCysPheThrAsnValTyrAlaAspSerPhe
  401 ValIleArgGlyAspGluValArgGlnIleAlaProGlyGlnThrGlyLysIleAlaAsp
  421 TyrAsnTyrLysLeuProAspAspPheThrGlyCysValIleAlaTrpAsnSerAsnAsn
  441 LeuAspSerLysValGlyGlyAsnTyrAsnTyrLeuTyrArgLeuPheArgLysSerAsn
  461 LeuLysProPheGluArgAspIleSerThrGluIleTyrGlnAlaGlySerThrProCys
  481 AsnGlyValGluGlyPheAsnCysTyrPheProLeuGlnSerTyrGlyPheGlnProThr
  501 AsnGlyValGlyTyrGlnProTyrArgValValValLeuSerPheGluLeuLeuHisAla
  521 ProAlaThrValCysGlyProLysLysSerThrAsnLeuValLysAsnLysCysValAsn
  541 PheAsnPheAsnGlyLeuThrGlyThrGlyValLeuThrGluSerAsnLysLysPheLeu
  561 ProPheGlnGlnPheGlyArgAspIleAlaAspThrThrAspAlaValArgAspProGln
  581 ThrLeuGluIleLeuAspIleThrProCysSerPheGlyGlyValSerValIleThrPro
  601 GlyThrAsnThrSerAsnGlnValAlaValLeuTyrGlnAspValAsnCysThrGluVal
  621 ProValAlaIleHisAlaAspGlnLeuThrProThrTrpArgValTyrSerThrGlySer
  641 AsnValPheGlnThrArgAlaGlyCysLeuIleGlyAlaGluHisValAsnAsnSerTyr
  661 GluCysAspIleProIleGlyAlaGlyIleCysAlaSerTyrGlnThrGlnThrAsnSer
  681 ProGlySerAlaSerSerValAlaSerGlnSerIleIleAlaTyrThrMetSerLeuGly
  701 AlaGluAsnSerValAlaTyrSerAsnAsnSerIleAlaIleProThrAsnPheThrIle
  721 SerValThrThrGluIleLeuProValSerMetThrLysThrSerValAspCysThrMet
  741 TyrIleCysGlyAspSerThrGluCysSerAsnLeuLeuLeuGlnTyrGlySerPheCys
  761 ThrGlnLeuAsnArgAlaLeuThrGlyIleAlaValGluGInAspLysAsnThrGlnGlu
  781 ValPheAlaGlnValLysGlnIleTyrLysThrProProIleLysAspPheGlyGlyPhe
  801 AsnPheSerGlnIleLeuProAspProSerLysProSerLysArgSerPheIleGluAsp
  821 LeuLeuPheAsnLysValThrLeuAlaAspAlaGlyPheIleLysGlnTyrGlyAspCys
  841 LeuGlyAspIleAlaAlaArgAspLeuIleCysAlaGInLysPheAsnGlyLeuThrVal
  861 LeuProProLeuLeuThrAspGluMetIleAlaGInTyrThrSerAlaLeuLeuAlaGly
  881 ThrIleThrSerGlyTrpThrPheGlyAlaGlyAlaAlaLeuGlnIleProPheAlaMet
  901 GlnMetAlaTyrArgPheAsnGlyIleGlyValThrGlnAsnValLeuTyrGluAsnGln
  921 LysLeuIleAlaAsnGlnPheAsnSerAlaIleGlyLysIleGInAspSerLeuSerSer
  941 ThrAlaSerAlaLeuGlyLysLeuGlnAspValValAsnGlnAsnAlaGInAlaLeuAsn
  961 ThrLeuValLysGlnLeuSerSerAsnPheGlyAlaIleSerSerValLeuAsnAspIle
  981 LeuSerArgLeuAspProProGluAlaGluValGlnIleAspArgLeuIleThrGlyArg
1001 LeuGlnSerLeuGlnThrTyrValThrGlnGlnLeuIleArgAlaAlaGluIleArgAla
1021 SerAlaAsnLeuAlaAlaThrLysMetSerGluCysValLeuGlyGlnSerLysArgVal
1041 AspPheCysGlyLysGlyTyrHisLeuMetSerPheProGlnSerAlaProHisGlyVal
1061 ValPheLeuHisValThrTyrValProAlaGInGluLysAsnPheThrThrAlaProAla
1081 IleCysHisAspGlyLysAlaHisPheProArgGluGlyValPheValSerAsnGlyThr
1101 HisTrpPheValThrGlnArgAsnPheTyrGluProGlnIleIleThrThrAspAsnThr
1121 PheValSerGlyAsnCysAspValValIleGlyIleValAsnAsnThrValTyrAspPro
1141 LeuGlnProGluLeuAspSerPheLysGluGluLeuAspLysTyrPheLysAsnHisThr
1161 SerProAspValAspLeuGlyAspIleSerGlyIleAsnAlaSerValValAsnIleGln
1181 LysGluIleAspArgLeuAsnGluValAlaLysAsnLeuAsnGluSerLeuIleAspLeu
1201 GlnGluLeuGlyLysTyrGluGlnTyrIleLysTrpProGlyGlySerGlyGlyTyrIle
1221 ProGluAlaProArgAspGlyGlnAlaTyrValArgLysAspGlyGluTrpValLeuLeu
1241 SerThrPheLeuGlyGlyGlyGlySerGlyGlyGlySerValSerGlyTrpArgLeuPhe
1261 LysLysIleSerGlyGlySerHisHisHisHisHisHisHisHis
β10-S-β10 nucleotide sequence
(SEQ ID NO: 23)
      1 ATGCGGCTTC CGGGTGCGAT GCCAGCTCTG GCCCTCAAAG GCGAGCTGCT GTTGCTGTCT
    61 CTCCTGTTAC TTCTGGAACC ACAGATCTCT CAGGGCGGTG GaGTGAGCGG CTGGCGGCTG
121 TTCAAGAAGA TTAGtGGTGG CGGAGGATCT GGAGGTGGAT CCtgcgtcaa tctgacaact
  181 cggactcagc tgccacctgc ttatactaat agcttcacca gaggcgtgta ctatcctgac
  241 aaggtgttta gaagctccgt gctgcactct acacaggatc tgtttctgcc attctttagc
  301 aacgtgacct ggttccacgc catccacgtg agcggcacca atggcacaaa gcggttcgac
  361 aatcccgtgc tgccttttaa cgatggcgtg tacttcgcct ctaccgagaa gagcaacatc
  421 atcagaggct ggatctttgg caccacactg gactccaaga cacagtctct gctgatcgtg
  481 aacaatgcca ccaacgtggt catcaaggtg tgcgagttcc agttttgtaa tgatcccttc
  541 ctgggcgtgt actatcacaa gaacaataag agctggatgg agtccgagtt tagagtgtat
  601 tctagcgcca acaactgcac atttgagtac gtgagccagc ctttcctgat ggacctggag
  661 ggcaagcagg gcaatttcaa gaacctgagg gagttcgtgt ttaagaatat cgacggctac
  721 ttcaaaatct actctaagca cacccccatc aacctggtgc gcgacctgcc tcagggcttc
  781 agcgccctgg agcccctggt ggatctgcct atcggcatca acatcacccg gtttcagaca
  841 ctgctggccc tgcacagaag ctacctgaca cccggcgact cctctagcgg atggaccgcc
  901 ggcgctgccg cctactatgt gggctacctc cagccccgga ccttcctgct gaagtacaac
  961 gagaatggca ccatcacaga cgcagtggat tgcgccctgg accccctgag cgagacaaag
1021 tgtacactga agtcctttac cgtggagaag ggcatctatc agacatccaa tttcagggtg
1081 cagccaaccg agtctatcgt gcgctttcct aatatcacaa acctgtgccc atttggcgag
1141 gtgttcaacg caacccgctt cgccagcgtg tacgcctgga ataggaagcg gatcagcaac
1201 tgcgtggccg actatagcgt gctgtacaac tccgcctctt tcagcacctt taagtgctat
1261 ggcgtgtccc ccacaaagct gaatgacctg tgctttacca acgtctacgc cgattctttc
1321 gtgatcaggg gcgacgaggt gcgccagatc gcccccggcc agacaggcaa gatcgcagac
1381 tacaattata agctgccaga cgatttcacc ggctgcgtga tcgcctggaa cagcaacaat
1441 ctggattcca aagtgggcgg caactacaat tatctgtacc ggctgtttag aaagagcaat
1501 ctgaagccct tcgagaggga catctctaca gaaatctacc aggccggcag caccccttgc
1561 aatggcgtgg agggctttaa ctgttatttc ccactccagt cctacggctt ccagcccaca
1621 aacggcgtgg gctatcagcc ttaccgcgtg gtggtgctga gctttgagct gctgcacgcc
1681 ccagcaacag tgtgcggccc caagaagtcc accaatctgg tgaagaacaa gtgcgtgaac
1741 ttcaacttca acggcctgac cggcacaggc gtgctgaccg agtccaacaa gaagttcctg
1801 ccatttcagc agttcggcag ggacatcgca gataccacag acgccgtgcg cgacccacag
1861 accctggaga tcctggacat cacaccctgc tctttcggcg gcgtgagcgt gatcacaccc
1921 ggcaccaata caagcaacca ggtggccgtg ctgtatcagg acgtgaattg taccgaggtg
1981 cccgtggcta tccacgccga tcagctgacc ccaacatggc gggtgtacag caccggctcc
2041 aacgtcttcc agacaagagc cggatgcctg atcggagcag agcacgtgaa caattcctat
2101 gagtgcgaca tcccaatcgg cgccggcatc tgtgcctctt accagaccca gacaaactct
2161 cccGgaagCg ccAgCagcgt ggcctcccag tctatcatcg cctataccat gtccctgggc
2221 gccgagaaca gcgtggccta ctctaacaat agcatcgcca tcccaaccaa cttcacaatc
2281 tctgtgacca cagagatcct gcccgtgtcc atgaccaaga catctgtgga ctgcacaatg
2341 tatatctgtg gcgattctac cgagtgcagc aacctgctgc tccagtacgg cagcttttgt
2401 acccagctga atagagccct gacaggcatc gccgtggagc aggataagaa cacacaggag
2461 gtgttcgccc aggtgaagca aatctacaag acccccccta tcaaggactt tggcggcttc
2521 aatttttccc agatcctgcc tgatccatcc aagccttcta agcggagctt tatcgaggac
2581 ctgctgttca acaaggtgac cctggccgat gccggcttca tcaagcagta tggcgattgc
2641 ctgggcgaca tcgcagccag ggacctgatc tgcgcccaga agtttaatgg cctgaccgtg
2701 ctgccacccc tgctgacaga tgagatgatc gcacagtaca caagcgccct gctggccggc
2761 accatcacat ccggatggac cttcggcgca ggagccgccc tccagatccc ctttgccatg
2821 cagatggcct ataggttcaa cggcatcggc gtgacccaga atgtgctgta cgagaaccag
2881 aagctgatcg ccaatcagtt taactccgcc atcggcaaga tccaggacag cctgtcctct
2941 acagccagcg ccctgggcaa gctccaggat gtggtgaatc agaacgccca ggccctgaat
3001 accctggtga agcagctgag cagcaacttc ggcgccatct ctagcgtgct gaatgacatc
3061 ctgagccggc tggacCCTCC Tgaggcagag gtgcagatcg accggctgat caccggccgg
3121 ctccagagcc tccagaccta tgtgacacag cagctgatca gggccgccga gatcagggcc
3181 agcgccaatc tggcagcaac caagatgtcc gagtgcgtgc tgggccagtc taagagagtg
3241 gacttttgtg gcaagggcta tcacctgatg tccttccctc agtctgcccc acacggcgtg
3301 gtgtttctgc acgtgaccta cgtgcccgcc caggagaaga acttcaccac agcccctgcc
3361 atctgccacg atggcaaggc ccactttcca agggagggcg tgttcgtgtc caacggcacc
3421 cactggtttg tgacacagcg caatttctac gagccccaga tcatcaccac agacaacacc
3481 ttcgtgagcg gcaactgtga cgtggtcatc ggcatcgtga acaataccgt gtatgatcca
3541 ctccagcccg agctggacag ctttaaggag gagctggata agtatttcaa gaatcacacc
3601 tcccctgacg tggatctggg cgacatcagc ggcatcaatg cctccgtggt gaacatccag
3661 aaggagatcg accgcctgaa cgaggtggct aagaatctga acgagagcct gatcgacctc
3721 caggagctgg gcaagtatga gcagtacatc aagtggcccG GaGGCAGCGG aGGCTACATC
3781 CCCGAGGCCC CtCGCGAtGG aCAGGCtTAC GTGCGCAAGG ACGGCGAGTG GGTGCTGCTG
3841 AGCACCTTCC TGGGCGGAGG AGGATCTGGA GGTGGATCCG TGAGCGGCTG GCGGCTGTTC
3901 AAGAAGATTA GCGGCGGAAG CCATCATCAC CACCATCATC ACCATTGA
β10-S-β10 amino acid sequence
(SEQ ID NO: 24)
      1 MetArgLeuProGlyAlaMetProAlaLeuAlaLeuLysGlyGluLeuLeuLeuLeuSer
    21 LeuLeuLeuLeuLeuGluProGlnIleSerGlnGlyGlyGlyValSerGlyTrpArgLeu
    41 PheLysLysIleSerGlyGlyGlyGlySerGlyGlyGlySerCysValAsnLeuThrThr
    61 ArgThrGlnLeuProProAlaTyrThrAsnSerPheThrArgGlyValTyrTyrProAsp
    81 LysValPheArgSerSerValLeuHisSerThrGlnAspLeuPheLeuProPhePheSer
  101 AsnValThrTrpPheHisAlaIleHisValSerGlyThrAsnGlyThrLysArgPheAsp
  121 AsnProValLeuProPheAsnAspGlyValTyrPheAlaSerThrGluLysSerAsnIle
  141 IleArgGlyTrpIlePheGlyThrThrLeuAspSerLysThrGlnSerLeuLeuIleVal
  161 AsnAsnAlaThrAsnValValIleLysValCysGluPheGInPheCysAsnAspProPhe
  181 LeuGlyValTyrTyrHisLysAsnAsnLysSerTrpMetGluSerGluPheArgValTyr
  201 SerSerAlaAsnAsnCysThrPheGluTyrValSerGlnProPheLeuMetAspLeuGlu
  221 GlyLysGlnGlyAsnPheLysAsnLeuArgGluPheValPheLysAsnIleAspGlyTyr
  241 PheLysIleTyrSerLysHisThrProIleAsnLeuValArgAspLeuProGInGlyPhe
  261 SerAlaLeuGluProLeuValAspLeuProIleGlyIleAsnIleThrArgPheGlnThr
  281 LeuLeuAlaLeuHisArgSerTyrLeuThrProGlyAspSerSerSerGlyTrpThrAla
  301 GlyAlaAlaAlaTyrTyrValGlyTyrLeuGlnProArgThrPheLeuLeuLysTyrAsn
  321 GluAsnGlyThrIleThrAspAlaValAspCysAlaLeuAspProLeuSerGluThrLys
  341 CysThrLeuLysSerPheThrValGluLysGlyIleTyrGlnThrSerAsnPheArgVal
  361 GlnProThrGluSerIleValArgPheProAsnIleThrAsnLeuCysProPheGlyGlu
  381 ValPheAsnAlaThrArgPheAlaSerValTyrAlaTrpAsnArgLysArgIleSerAsn
  401 CysValAlaAspTyrSerValLeuTyrAsnSerAlaSerPheSerThrPheLysCysTyr
  421 GlyValSerProThrLysLeuAsnAspLeuCysPheThrAsnValTyrAlaAspSerPhe
  441 ValIleArgGlyAspGluValArgGlnIleAlaProGlyGlnThrGlyLysIleAlaAsp
  461 TyrAsnTyrLysLeuProAspAspPheThrGlyCysValIleAlaTrpAsnSerAsnAsn
  481 LeuAspSerLysValGlyGlyAsnTyrAsnTyrLeuTyrArgLeuPheArgLysSerAsn
  501 LeuLysProPheGluArgAspIleSerThrGluIleTyrGlnAlaGlySerThrProCys
  521 AsnGlyValGluGlyPheAsnCysTyrPheProLeuGlnSerTyrGlyPheGlnProThr
  541 AsnGlyValGlyTyrGlnProTyrArgValValValLeuSerPheGluLeuLeuHisAla
  561 ProAlaThrValCysGlyProLysLysSerThrAsnLeuValLysAsnLysCysValAsn
  581 PheAsnPheAsnGlyLeuThrGlyThrGlyValLeuThrGluSerAsnLysLysPheLeu
  601 ProPheGlnGlnPheGlyArgAspIleAlaAspThrThrAspAlaValArgAspProGln
  621 ThrLeuGluIleLeuAspIleThrProCysSerPheGlyGlyValSerValIleThrPro
  641 GlyThrAsnThrSerAsnGlnValAlaValLeuTyrGlnAspValAsnCysThrGluVal
  661 ProValAlaIleHisAlaAspGlnLeuThrProThrTrpArgValTyrSerThrGlySer
  681 AsnValPheGlnThrArgAlaGlyCysLeuIleGlyAlaGluHisValAsnAsnSerTyr
  701 GluCysAspIleProIleGlyAlaGlyIleCysAlaSerTyrGlnThrGlnThrAsnSer
  721 ProGlySerAlaSerSerValAlaSerGlnSerIleIleAlaTyrThrMetSerLeuGly
  741 AlaGluAsnSerValAlaTyrSerAsnAsnSerIleAlaIleProThrAsnPheThrIle
  761 SerValThrThrGluIleLeuProValSerMetThrLysThrSerValAspCysThrMet
  781 TyrIleCysGlyAspSerThrGluCysSerAsnLeuLeuLeuGlnTyrGlySerPheCys
  801 ThrGlnLeuAsnArgAlaLeuThrGlyIleAlaValGluGlnAspLysAsnThrGlnGlu
  821 ValPheAlaGlnValLysGlnIleTyrLysThrProProIleLysAspPheGlyGlyPhe
  841 AsnPheSerGlnIleLeuProAspProSerLysProSerLysArgSerPheIleGluAsp
  861 LeuLeuPheAsnLysValThrLeuAlaAspAlaGlyPheIleLysGlnTyrGlyAspCys
  881 LeuGlyAspIleAlaAlaArgAspLeuIleCysAlaGInLysPheAsnGlyLeuThrVal
  901 LeuProProLeuLeuThrAspGluMetIleAlaGlnTyrThrSerAlaLeuLeuAlaGly
  921 ThrIleThrSerGlyTrpThrPheGlyAlaGlyAlaAlaLeuGlnIleProPheAlaMet
  941 GlnMetAlaTyrArgPheAsnGlyIleGlyValThrGlnAsnValLeuTyrGluAsnGln
  961 LysLeuIleAlaAsnGlnPheAsnSerAlaIleGlyLysIleGInAspSerLeuSerSer
  981 ThrAlaSerAlaLeuGlyLysLeuGlnAspValValAsnGlnAsnAlaGInAlaLeuAsn
1001 ThrLeuValLysGlnLeuSerSerAsnPheGlyAlaIleSerSerValLeuAsnAspIle
1021 LeuSerArgLeuAspProProGluAlaGluValGlnIleAspArgLeuIleThrGlyArg
1041 LeuGlnSerLeuGlnThrTyrValThrGlnGlnLeuIleArgAlaAlaGluIleArgAla
1061 SerAlaAsnLeuAlaAlaThrLysMetSerGluCysValLeuGlyGlnSerLysArgVal
1081 AspPheCysGlyLysGlyTyrHisLeuMetSerPheProGlnSerAlaProHisGlyVal
1101 ValPheLeuHisValThrTyrValProAlaGInGluLysAsnPheThrThrAlaProAla
1121 IleCysHisAspGlyLysAlaHisPheProArgGluGlyValPheValSerAsnGlyThr
1141 HisTrpPheValThrGlnArgAsnPheTyrGluProGlnIleIleThrThrAspAsnThr
1161 PheValSerGlyAsnCysAspValValIleGlyIleValAsnAsnThrValTyrAspPro
1181 LeuGlnProGluLeuAspSerPheLysGluGluLeuAspLysTyrPheLysAsnHisThr
1201 SerProAspValAspLeuGlyAspIleSerGlyIleAsnAlaSerValValAsnIleGln
1221 LysGluIleAspArgLeuAsnGluValAlaLysAsnLeuAsnGluSerLeuIleAspLeu
1241 GlnGluLeuGlyLysTyrGluGlnTyrIleLysTrpProGlyGlySerGlyGlyTyrIle
1261 ProGluAlaProArgAspGlyGlnAlaTyrValArgLysAspGlyGluTrpValLeuLeu
1281 SerThrPheLeuGlyGlyGlyGlySerGlyGlyGlySerValSerGlyTrpArgLeuPhe
1301 LysLysIleSerGlyGlySerHisHisHisHisHisHisHisHisEnd
```

### REFERENCES

1. Tang, Y., Schmitz, J. E., Persing, D. H. & Stratton, C. W. Laboratory Diagnosis of COVID-19: Current Issues and Challenges. J. Clin. Microbiol. 58, e00512-20 (2020).
2. Peeling, R. W. et al. Serology testing in the COVID-19 pandemic response. Lancet Infect. Dis. 20, e245—e249 (2020).
3. Huang, A. T. et al. A systematic review of antibody mediated immunity to coronaviruses: kinetics, correlates of protection, and association with severity. Nat. Commun. 11, 4704 (2020).
4. Lisboa Bastos, M. et al. Diagnostic accuracy of serological tests for covid-19: Systematic review and meta-analysis. BMJ 370, (2020).
5. Andryukov, B. G. Six decades of lateral flow immunoassay: From determining metabolic markers to diagnosing covid-19. AIMS Microbiol. 6, 280-304 (2020).
6. Whitman, J. D. et al. Evaluation of SARS-CoV-2 serology assays reveals a range of test performance. Nat. Biotechnol. 38, 1174-1183 (2020).
7. Adams, E. R. et al. Antibody testing for COVID-19: A report from the National COVID Scientific Advisory Panel. Wellcome Open Res. 5, 139 (2020).
8. Amanat, F. et al. A serological assay to detect SARS-CoV-2 seroconversion in humans. Nat. Med. 26, 1033-1036 (2020).
9. Stadlbauer, D. et al. SARS-CoV-2 Seroconversion in Humans: A Detailed Protocol for a Serological Assay, Antigen Production, and Test Setup. Curr. Protoc. Microbiol. 57, 1-15 (2020).
10. Bryan, A. et al. Performance Characteristics of the Abbott Architect SARS-CoV-2 IgG Assay and Seroprevalence in Boise, Idaho. J. Clin. Microbiol. 58, 4-11 (2020).
11. Rhoads, D. D. et al. Comparison of abbott id now, diasorin simplexa, and CDC fda emergency use authorization methods for the detection of sars-cov-2 from nasopharyngeal and nasal swabs from individuals diagnosed with covid-19. J. Clin. Microbiol. 58, 17-18 (2020).
12. Package insert of Elecsys Anti-SARS-CoV-2. (2020). doi:10.1093/cid/ciaa344
13. Ainsworth, M. et al. Performance characteristics of five immunoassays for SARS-CoV-2: a head-to-head benchmark comparison. Lancet Infect. Dis. 3099, 1-11 (2020).
14. Yao, Z., Petschnigg, J., Ketteler, R. & Stagljar, I. Application guide for omics approaches to cell signaling. Nat. Chem. Biol. 11, 387-397 (2015).
15. Snider, J. et al. Fundamentals of protein interaction network mapping. Mol. Syst. Biol. 11, 848-848 (2015).
16. Michnick, S. W., Ear, P. H., Manderson, E. N., Remy, I. & Stefan, E. Universal strategies in research and drug discovery based on protein-fragment complementation assays. Nat. Rev. Drug Discov. 6, 569-582 (2007).
17. Remy, I. & Michnick, S. W. A highly sensitive protein-protein interaction assay based on Gaussia luciferase. Nat. Methods 3, 977-979 (2006).
18. Azad, T., Tashakor, A. & Hosseinkhani, S. Split-luciferase complementary assay: applications, recent developments, and future perspectives. Anal. Bioanal. Chem. 406, 5541-5560 (2014).
19. Ohmuro-Matsuyama, Y. & Ueda, H. Protein-Protein Interaction Assays Using Split-NanoLuc. in Bioluminescence-Analytical Applications and Basic Biology (IntechOpen, 2019). doi:10.5772/intechopen.86122
20. Dixon, A. S., Kim, S. J., Baumgartner, B. K., Krippner, S. & Owen, S. C. A Tri-part Protein Complementation System Using Antibody-Small Peptide Fusions Enables Homogeneous Immunoassays. Sci. Rep. 7, 1-13 (2017).
21. Ohmuro-Matsuyama, Y. & Ueda, H. Homogeneous Noncompetitive Luminescent Immunodetection of Small Molecules by Ternary Protein Fragment Complementation. Anal. Chem. 90, 3001-3004 (2018).
22. Kruger, N. J. Detection of Polypeptides on Immunoblots Using Enzyme-Conjugated or Radiolabeled Secondary Ligands. in The Protein Protocols Handbook (ed. Walker, J. M.) 405-414 (2002).
23. Zhou, P. et al. A pneumonia outbreak associated with a new coronavirus of probable bat origin. Nature 579, 270-273 (2020).
24. Ju, B. et al. Human neutralizing antibodies elicited by SARS-CoV-2 infection. Nature 584, 115-119 (2020).
25. Yuan, M. et al. A highly conserved cryptic epitope in the receptor binding domains of SARS-CoV-2 and SARS-CoV. Science (80-.). 368, 630-633 (2020).
26. Cassidy, J. T. & Nordby, G. L. Human serum immunoglobulin concentrations: Prevalence of immunoglobulin deficiencies. J. Allergy Clin. Immunol. 55, 35-48 (1975).
27. Gonzalez-Quintela, A. et al. Serum levels of immunoglobulins (IgG, IgA, IgM) in a general adult population and their relationship with alcohol consumption, smoking and common metabolic abnormalities. Clin. Exp. Immunol. 151, 42-(2008).
28. Hartman, H., Wang, Y., Schroeder, H. W. & Cui, X. Absorbance summation: A novel approach for analyzing high-throughput ELISA data in the absence of a standard. PLoS One 13, 1-12 (2018).
29. Tan, C. W. et al. A SARS-CoV-2 surrogate virus neutralization test based on antibody-mediated blockage of ACE2-spike protein-protein interaction. Nat. Biotechnol. 38, 1073-1078 (2020).
30. Mendoza, E. J., Manguiat, K., Wood, H. & Drebot, M. Two Detailed Plaque Assay Protocols for the Quantification of Infectious SARS-CoV-2. Curr. Protoc. Microbiol. 57, 1-15 (2020).
31. Wang, S., Sakhatskyy, P., Chou, T.-H. W. & Lu, S. Assays for the assessment of neutralizing antibody activities against Severe Acute Respiratory Syndrome (SARS) associated coronavirus (SCV). J. Immunol. Methods 301, 21-30 (2005).
32. Kim, D. K. et al. A comprehensive, flexible collection of SARS-CoV-2 coding regions. G3 Genes, Genomes, Genet. 10, 3399-3402 (2020).
33. Abe, K. T. et al. A simple protein-based surrogate neutralization assay for SARS-CoV-2. JCI insight 5, (2020).
33. Zohar, T. et al. Compromised humoral functional evolution tracks with SARSCoV-2 mortality. Cell 183, 1508-1519.e12 (2020).
34. Isho, B. et al. Persistence of serum and saliva antibody responses to SARSCoV-2 spike antigens in COVID-19 patients. Sci. Immunol. 5, eabe5511 (2020).
35. Premkumar, L. et al. The receptor-binding domain of the viral spike protein is an immunodominant and highly specific target of antibodies in SARS-CoV-2 patients. Sci. Immunol. 5, eabc8413 (2020).
36. Long, Q. X. et al. Antibody responses to SARS-CoV-2 in patients with COVID-19. Nat. Med. 26,845-848 (2020).
37. Guo, L. et al. Profiling early humoral response to diagnose novel coronavirus disease (COVID-19). Clin. Infect. Dis. 71,778-785 (2020).

Although various embodiments of the disclosure have been described and illustrated, it will be apparent to those skilled in the art in light of the present description that numerous modifications and variations can be made. The scope of the invention is defined more particularly in the appended claims.

1. A serological detection system for detecting immunoglobulins (Ig) of a specific isotype against a target antigen in a sample, the serological detection system comprising: (a) a first probe having a first tag, the first probe having general binding affinity for all Ig of the same isotype as the specific Ig isotype in the sample, (b) a second probe having a second tag, the second probe being the target antigen of the specific Ig isotype in the sample, (c) a third tag, the third tag having binding affinity for the first tag and for the second tag so as to form a reporter complex in the presence of the specific IgG antibodies, and (d) a suitable substrate that generates an optically detectable signal in the presence of the reporter complex.

2. (canceled)

3. (canceled)

4. The serological detection system of claim 1, wherein the first probe recognizes immunoglobulin G (IgG) without cross-reactivity with other Ig isotypes.

5. The serological detection system of claim 1, wherein the first probe is a protein G, or a suitable domain thereof.

6. The serological detection system according of claim 1, wherein the first probe is a protein G of a Streptococcus sp, a C1 domain of Streptococcus sp, a C2 domain of Streptococcus sp or a C3 domain of protein G of Streptococcus sp.

7. (canceled)

8. (canceled)

9. The serological detection system of claim 1, wherein the first probe recognizes immunoglobulin M (IgM) without cross-reactivity with other Ig isotypes or the first probe recognizes immunoglobulin A (IgA) without cross-reactivity with other Ig isotopes.

10. The serological detection system of claim 1, wherein the third tag is a Δ11S peptide of a nanoluciferase.

11. The serological system of claim 1, wherein the first probe is a protein G or a suitable protein G domain for general detection of IgGs in the sample, and the first tag is a β9 peptide of a nanoluciferase, the second probe is the target antigen of the specific IgG isotype, the second tag is a β10 peptide of the nanoluciferase, the third tag is a Δ11S of the nanoluciferase NanoLuc or a fragment thereof, and the substrate is a reagent that generates the optically detectable signal in the presence of the nanoluciferase.

12. The serological system of claim 1, wherein the first probe is a protein G or a suitable protein G domain for general detection of IgGs in the sample, and the first tag is a β10 peptide of a nanoluciferase, the second probe is the target antigen of the specific IgG isotype, the second tag is a 139 peptide of the nanoluciferase, the third tag is a Δ11S of the nanoluciferase NanoLuc or a fragment thereof, and the substrate is a reagent that generates the optically detectable signal in the presence of the nanoluciferase.

13. (canceled)

14. (canceled)

15. The serological system of claim 11, wherein the β9 peptide is included at the N-terminus of the protein G or of the suitable protein G domain, and the 1310 peptide is included at both the N-terminus and the C-terminus of the protein characteristic of the target.

16. The serological system of claim 1, wherein the first probe is an anti IgM antibody for general detection of IgMs in the sample, and the first tag is a 139 peptide of a nanoluciferase, the second probe is the target antigen of the specific IgM isotype, the second tag is a 1310 peptide of the nanoluciferase NanoLuc, the third tag is a Δ11S of the nanoluciferase NanoLuc or a fragment thereof, and the substrate is a reagent that generates the optically detectable signal in the presence of the nanoluciferase NanoLuc.

17. The serological system of claim 1, wherein the target antigen is from an infectious pathogen, or wherein the target antigen is an autoimmune antigen and the specific Ig isotype are autoantibodies.

18. (canceled)

19. The serological system of claim 17, wherein the target antigen is the infectious pathogen, and wherein the pathogen is severe acute respiratory syndrome coronavirus 2 (SARS-CoV-2).

20. The serological system of claim 19, wherein the second probe is a spike (S) protein of SARS-CoV-2 or a fragment thereof, or a receptor binding protein of a spike protein of SARS-CoV-2.

21. (canceled)

22. (canceled)

23. The serological system of claim 1, wherein a strength of the optically detectable signal correlates with the amount of the specific Ig isotype in the sample, thereby quantitating the specific Ig isotype against the target antigen in the sample.

24. A serological method of detecting the presence of specific Ig isotype against a target antigen in a sample of a subject, the method comprising: (a) reacting the sample with the serological system of claim 1, and (b) exposing the sample to an apparatus that detects the optically detectable signal in the sample, wherein detection of the optically detectable signal in the sample is indicative of the presence of the specific Ig isotype against the target antigen in the sample, and wherein the optically detectable signal has a strength, and the strength of the optically detectable signal is quantifiable and correlates with the amount of the specific Ig isotype against the target antigen in the sample.

25. (canceled)

26. The serological method of claim 24, wherein the strength of the optically detectable signal detected in the sample is compared to strength of optically detectable signal detected from a control sample devoid of the specific Ig isotype (negative control), and when the strength of optically detectable signal from the sample is greater than the strength of the optically detectable signal from the negative control is indicative of the presence of the specific Ig isotype in the sample.

27. (canceled)

28. The serological method of claim 24, wherein the target antigen is from an infectious pathogen and wherein pathogen is a bacterium, a fungus, a virus, a yeast, algae or a protozoan, or wherein the target antigen is an autoimmune antigen and the specific Ig isotype are autoantibodies.

29. The serological method of claim 28, wherein the pathogen is severe acute respiratory syndrome coronavirus 2 (SARS-CoV-2), and wherein the second probe is a spike (S) protein of SARS-CoV-2 or a fragment thereof, or the second probe is a receptor binding protein of a spike protein of SARS-CoV-2.

30-58. (canceled)
